(19) United States
(12) Patent Application Publication
(10) Pub. No.: US 2020/0129435 A1
(43) Pub. Date: Apr. 30, 2020

(54) COMPOSITION FOR DELIVERING A THERAPEUTIC AGENT AND METHODS FOR MAKING AND USING

(71) Applicants: UVic Industry Partnerships Inc., Victoria (CA); The University of British Columbia, Vancouver (CA)

(72) Inventors: Mohsen Akbari, Victoria (CA); Brian Toyota, Vancouver (CA)

(73) Assignees: UVic Industry Partnerships Inc., Victoria (CA); The University of British Columbia, Vancouver (CA)

(21) Appl. No.: 16/169,591

(22) Filed: Oct. 24, 2018

(51) Int. Cl. A61K 9/16; A61K 45/06; A61P 35/00

(52) U.S. Cl. CPC A61K 9/1694; A61K 9/1658; A61K 9/1652; A61P 35/00; A61K 9/1635; A61K 45/06; A61K 9/1647

(57) ABSTRACT

Disclosed herein are embodiments of a composition comprising a matrix, a plurality of microparticles and a therapeutic agent. The therapeutic agent may be encapsulated in the microparticles, and/or the microparticles may be dispersed in the matrix. The composition may be a mesh or a sheet, and may further comprise a second therapeutic agent, and optionally a second microparticle and/or a second matrix. In particular embodiments, the matrix is a hydrogel. Methods for making and using the composition are also disclosed, and in certain embodiments, the composition is useful for treating cancer, such as brain cancer.

### FIELD

This disclosure concerns a composition comprising a matrix, a plurality of microparticles and a therapeutic agent, and methods for making and using the composition, the composition being useful for delivering the therapeutic agent.

### BACKGROUND

There are many benefits to delivering a therapeutic agent directly to a target and/or being able to control the release of that agent to the target. Such benefits may include reducing unwanted side effects such as off-target actions, reducing delivery amounts, and providing sustained or delayed release of a therapeutic agent. Certain targets, such as targets in the brain or vascular system, have fluid present at the target, and may have fluid flowing past the target. Delivery of a therapeutic agent at such sites may be problematic because the fluids with disperse and/or wash away the therapeutic agent. And delivery of the agent to the fluid may result in systemic administration that leads to side effects from the therapeutic agent and increased costs.

### SUMMARY

Disclosed herein is a composition comprising a plurality of microparticles within a matrix and a therapeutic agent encapsulated within the microparticles. The microparticles may have a microparticle diameter of from 1 μm to 250 μm. The matrix may be a non-liquid matrix. The matrix and the microparticles independently may be a hydrogel, a synthetic material, or a combination thereof, and in some embodiments, the matrix and the microparticles are different compositions. Hydrogels suitable for use in either the matrix, the microparticle, or both, include, but are not limited to, alginate, gelatin, agarose, hyaluronic acid, gelatin methacryloyl, chitosan, elastin, collage, polyethylene glycol, or a combination thereof. And synthetic materials suitable for use in either the matrix, the microparticle, or both, include, but are not limited to, poly lactic-co-glycolic acid (PLGA), polylactic acid, polycaprolactone (PCL), nanosilicates, polyvinyl alcohol, poly(n-isopropylacrylamide), or a combination thereof. In certain embodiments, the matrix comprises alginate, gelatin methacryloyl, or a combination thereof, the microparticles comprise PLGA, PCL, or a combination thereof, or both.

The therapeutic agent may comprise an anticancer drug, antiviral, antibiotic, antifungal agent, anti-parasitic, anti-inflammatory agent, pain killer, growth factor, antibody, peptide, cytokine, chemokine, immunomodulatory agent, radioactive composition, clotting aid, or a combination thereof. In particular embodiments, the therapeutic agent comprises an anticancer agent, and may comprise all-trans retinoic acid, temozolomide, doxorubicin; paclitaxel, 5-fluorouracil, doxurubicin hydrochloride, docetaxel, epirubicin, cisplatin, carboplatin, simvastatin, bevacizumab, ranibizumab, aflibercept, bradikinin, epidermal growth factor, vascular growth factor, interleukin 8, or a combination thereof.

In some embodiments, the composition is a mesh, and the matrix forms fibers that form the mesh. The fibers may have a fiber diameter of from greater than 50 to 1000 μm, and/or may be cross-linked, such as by exposure to UV radiation, calcium ions, or a temperature change.

In certain embodiments, the composition is a mesh composition comprising an alginate matrix with PLGA microparticles dispersed within, the PLGA microparticles encapsulating temozolomide. And in other embodiments, the composition is a mesh composition comprising an alginate or alginate/GelMA matrix with PCL microparticles dispersed within, the PCL microparticles encapsulating all-trans retinoic acid.

In any embodiments, the composition may comprise a first matrix comprising a first plurality of microparticles within the first matrix, and a first therapeutic agent encapsulated within the first microparticles; and a second matrix comprising a second plurality of microparticles within the second matrix, and a second therapeutic agent encapsulated within the second microparticles. And the first matrix and the second matrix together may form a mesh.

Also disclosed is a method for making the disclosed composition. The method may comprise forming a first mixture comprising the therapeutic agent and a polymer such that microparticles encapsulating the therapeutic agent form in the first mixture; and forming a second mixture comprising the plurality of microparticles and a matrix to form a composition comprising therapeutic agent-loaded microparticles dispersed within the matrix. The method may further comprise forming fibers comprising the second mixture, and cross-linking the fibers to form a mesh composition.

Additionally, disclosed herein is a method of using the disclosed composition. The method may be a method for treating a cancer, such as a solid tumor cancer. The method may comprise locating the disclosed composition, such as a mesh composition, adjacent to or onto a cancer cell, such as a solid tumor cancer cell. The cancer cell may be in a subject. The cancer cell may be a breast, lung, prostate, colon, brain, uterus, pancreas, skin, or liver cancer cell, and in certain embodiments, the cancer cell is a glioblastoma multiforme cancer cell.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic diagram illustrating one embodiment of a general method for making the disclosed composition using a co-axial flow 3D-printing system.

FIG. 2 provides digital images of a mesh made from the disclosed composition.

FIG. 3 is a digital microscope image of the mesh from FIG. 2.

FIG. 4 is a schematic representation illustrating one application of a mesh made from the disclosed composition implanted in a brain.

FIG. 5 are SEM images of ATRA-loaded and blank PCL microspheres.

FIG. 6 are SEM images of individual ATRA loaded particles showing their surface morphology.

FIG. 7 is a graph of frequency versus diameter, illustrating the size distribution of ATRA-loaded and blank PCL microspheres.

FIG. 8 is a graph of percent released drug versus time, illustrating the different release rates of ATRA-loaded microspheres in a release medium alone or when dispersed in a hydrogel.

FIG. 9 is a graph of fiber diameter versus the low-viscosity bioink flow rate, illustrating how bioink flow rate affects fiber diameter during 3-D printing.

FIG. 10 is a graph of fiber diameter versus printing speed, illustrating how the fiber diameter varies with printing speed during 3-D printing.

FIG. 11 provides digital images illustrating that increasing the bioink flow rate results in an increase in fiber diameter.

FIG. 12 provides digital images illustrating that printing with higher speeds results in a decrease in the fiber diameter.

FIG. 13 is a plot of extrusion temperature versus GelMA concentration, illustrating how the printability of the low-viscosity bioink varies with respect to GelMA concentration and extrusion temperature.

FIG. 14 provides digital microscope images comparing the results of printing under printable and substantially non-printable conditions.

FIG. 15 is a schematic diagram illustrating the drug release scaffold that was modelled in COMSOL 5.1 by considering a repeated segment of the scaffold in the medium.

FIG. 16 is a schematic representation of the mesh that was modelled for numerical simulation of drug diffusion to the medium.

FIG. 17 is a graph of flux versus time, illustrating the results from an experimental release study that was used to determine the diffusive flux from the scaffold to medium at their interface.

FIG. 18 is a schematic representation of the distribution of ATRA in different heights of the medium after 24 hours, derived from numerical simulation.

FIG. 19 is a graph of concentration versus height, illustrating the concentration of ATRA in different locations of medium in different time points from 12 hours to 10 days.

FIG. 20 is a graph of concentration versus time, illustrating the average concentration of ATRA against time for different porosity of the scaffold.

FIG. 21 is a graph of cell population versus time, illustrating the effect of free ATRA in the cell medium as determined by a cell counting assay, and showing that the presence of ATRA reduced the proliferation of U-87 MG cells after 48, 72, and 96 hours of exposure.

FIG. 22 is a graph of cell viability versus time, illustrating the results from exposing U-87 MG cells to two different concentrations of free ATRA, and showing that 20 μM and 40 μM ATRA led to 77% and 72% viability after 96 hours, respectively.

FIG. 23 is a graph of cell viability versus time, illustrating the effect of ATRA-loaded hydrogel with two different morphologies, gel sheet and mesh, as determined by exposing U-87 MG cells to the drug.

FIG. 24 provides digital bright field microscope images of U-87 MG cells treated with different forms of ATRA, illustrating the lower cell confluence in wells treated with ATRA, and showing the cell shrinkage resulting from free ATRA, and the apoptotic cell morphology associated with ATRA-loaded mesh. Scale bars: 100 μm.

FIG. 25 is a graph of apoptosis rate expressed as percent sub-G1 population versus treatment, illustrating that U-87 MG cells did not show a considerable apoptosis level when exposed directly to ATRA, with simvastatin treatment used as a positive control condition.

FIG. 26 is a graph of apoptosis rate expressed as percent sub-G1 population versus treatment, illustrating that hydrogel meshes laden with ATRA-loaded PCL microspheres induced considerable apoptosis in U-87 MG cells after 48 hours, with simvastatin treatment used as a positive control condition.

FIG. 27 are graphs of cell population versus florescence intensity obtained from flow cytometry, illustrating the cell population distribution of cell exposed to free ATRA, compared to control and simvastatin-exposed cells.

FIG. 28 are graphs of cell population versus florescence intensity obtained from flow cytometry, illustrating the cell population distributions of cell exposed to ATRA-loaded hydrogel, compared to control, simvastatin-exposed and blank hydrogel-exposed cells.

FIG. 29 is an SEM image a hydrogel mesh loaded with PCL microspheres.

FIG. 30 provides SEM images of the hydrogel mesh of FIG. 29 at higher magnifications to show the presence of the microspheres in the hydrogel fibers.

FIG. 31 is a fluorescent microscopy image of a mesh comprising one embodiment of the disclosed hydrogel composition prepared using 0.2 mg ATRA-loaded PCL microspheres per 1 mL of hydrogel, illustrating the substantially uniform dispersion of microspheres inside the hydrogel structures.

FIG. 32 is a fluorescent microscopy image of a mesh comprising one embodiment of the disclosed hydrogel composition prepared using 0.5 mg ATRA-loaded PCL microspheres per 1 mL of hydrogel, illustrating the substantially uniform dispersion of microspheres inside the hydrogel structures.

FIG. 33 is a fluorescent microscopy image of a mesh comprising one embodiment of the disclosed hydrogel composition prepared using 2.0 mg ATRA-loaded PCL microspheres per 1 mL of hydrogel, illustrating the substantially uniform dispersion of microspheres inside the hydrogel structures.

FIG. 34 provides SEM images of blank PLGA microspheres prepared with 1.25%, 5%, and 10% PLGA concentrations, illustrating the morphology of the microspheres.

FIG. 35 provides SEM images of TMZ-loaded PLGA microspheres prepared with 1.25%, 5%, and 10% PLGA concentrations, illustrating the morphology of the microspheres.

FIG. 36A is a graph of frequency (%) versus size, illustrating the size distribution of blank PLGA microspheres made at a 1.25% PLGA concentration.

FIG. 36B is a graph of frequency (%) versus size, illustrating the size distribution of TMZ-loaded PLGA microspheres made at a 1.25% PLGA concentration.

FIG. 37A is a graph of frequency (%) versus size, illustrating the size distribution of blank PLGA microspheres made at a 5% PLGA concentration.

FIG. 37B is a graph of frequency (%) versus size, illustrating the size distribution of TMZ-loaded PLGA microspheres made at a 5% PLGA concentration.

FIG. 38A is a graph of frequency (%) versus size, illustrating the size distribution of blank PLGA microspheres made at a 10% PLGA concentration.

FIG. 38B is a graph of frequency (%) versus size, illustrating the size distribution of TMZ-loaded PLGA microspheres made at a 10% PLGA concentration.

FIG. 39 is a graph of average size versus microsphere type, illustrating the average size of blank and TMZ-loaded PLGA microspheres prepared with 1.25%, 5%, and 10% PLGA concentrations.

FIG. 40 is a digital image of one embodiment of the disclosed hydrogel composition fabricated as a mesh.

FIG. 41 is a digital image of the hydrogel mesh from FIG. 40 illustrating a different viewing angle.

FIG. 42 is an SEM image of one embodiment of the disclosed hydrogel composition fabricated as a mesh.

FIG. 43 is a graph of fiber dimension and surface-to-volume ratio versus pressure, illustrating how higher pressures on the nozzle resulted in larger fiber diameters and smaller surface-to-volume ratio.

FIG. 44 provides microscope images illustrating alginate meshes printed with 40 kPa pressure, 80 kPa pressure, and 120 kPa nozzle pressure.

FIG. 45 is a graph of fiber dimension and surface-to-volume ratio versus printing speed, illustrating how higher printing speeds resulted in decreased fiber diameters and larger surface-to-volume ratio.

FIG. 46 provides microscope images illustrating alginate meshes printed with 250 mm min⁻¹, 350 mm min⁻¹, and 450 mm min⁻¹ printing speeds.

FIG. 47 is a graph of fiber diameter versus microsphere concentration, illustrating that the fiber diameter increased at higher microsphere densities.

FIG. 48 provides microscope images illustrating alginate meshes printed with 1 mg mL⁻¹, 3 mg mL⁻¹, and 6 mg mL⁻¹ microsphere concentration.

FIG. 49 is a graph of percent release versus time, illustrating the effect of PLGA concentration on the release profile of TMZ from PLGA microspheres, and demonstrating that TMZ-loaded PLGA microspheres prepared with higher PLGA concentrations showed lower initial burst release and slower overall release rate, possibly due to their smaller surface-to-volume ratio.

FIG. 50 is a graph of percent release versus time, illustrating that incorporation of microspheres prepared with 5% PLGA concentration within alginate fibers resulted in a more sustained TMZ release.

FIG. 51 is a graph of percent release versus time, illustrating that increasing the fiber diameter resulted in slower TMZ release kinetics, possibly due to a lower surface-to-volume ratio.

FIG. 52 is a graph of viability versus time, illustrating the cytotoxicity of different concentrations of free TMZ in U87 glioblastoma cells and showing that by increasing the TMZ concentration the cytotoxic effects increased.

FIG. 53 is a graph of viability versus time, illustrating the cytotoxicity of blank and microsphere-loaded mesh to U87 glioblastoma cells, and showing that cell viability decreased considerably after 72 hours treatment with drug/microsphere-loaded mesh with a TMZ concentration of 100 μM.

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood to one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

Gel: A mass composed of at least two components, one of which forms a three-dimensional network by virtue of covalent or noncovalent bonding (chemical and physical gels, respectively) in the medium of the other component (typically a liquid), wherein the minimum amount of the liquid is sufficient for ensuring the elastic properties of the gel. A gel can sustain shear stress and also undergo liquid-solid transitions.

Hydrogel: A substance formed when a hydrophilic organic or inorganic polymer is cross-linked via covalent, ionic, and/or hydrogen bonds to create a three-dimensional open-lattice structure which takes up water molecules to form a gel.

### II. Composition

Disclosed herein are embodiments of a composition comprising a matrix, a plurality of microparticles dispersed into the matrix, and a therapeutic agent encapsulated in the microparticles. Typically, the microparticles are formed from a material that is different to that of the matrix.

A. Matrix

In some embodiments, the matrix is biodegradable, but in other embodiments, the matrix is not biodegradable. The matrix may be permeable to water and aqueous solutions, such as biological fluids. And in some embodiments, the matrix is flexible and in certain embodiments, the composition forms a mesh or sheet that can be formed to substantially conform to the shape of to a desire area, such as a tumor, wound, or particular part of a body, such as the brain.

The matrix may be a non-liquid matrix and/or non-gaseous matrix. In some embodiments, the matrix is a solid or semi-solid matrix, such as a gel. The matrix may comprise a hydrogel, a synthetic material, or a combination thereof. Hydrogels suitable for use in the disclosed composition include any hydrogel that can be formed into a desired shape, such as a mesh, while remaining flexible. In some embodiments, the hydrogel is a biodegradable hydrogel, but in other embodiments, the hydrogel is not a biodegradable hydrogel. In certain embodiments, the hydrogel is suitable for use in a 3-D printing process. Exemplary hydrogels include, but are not limited to, alginate, gelatin, agarose, hyaluronic acid, gelatin methacryloyl (GelMA), chitosan, elastin, collage, polyethylene glycol, or a combination thereof. The matrix may comprise one hydrogel or it may comprise two or more hydrogels, such as 2, 3, 4, 5 or more hydrogels. In some embodiments, the matrix comprises a first hydrogel and a second hydrogel, in a ratio relative to each other, of from 1%:99% to 99%:1% w/w, such as from 10%:90% to 90%:10%, from 20%:80% to 80%:20%, from 30%:70% to 70%:30%, from 40%:60% to 60%:40%, or from 50%:50%.

In some embodiments, the matrix comprises, consists essentially of, or consists of, a hydrogel. And in particular embodiments, the matrix comprises, consists essentially of, or consists of, alginate, gelatin methacryloyl, or a combination thereof.

In other embodiments, the matrix comprises a synthetic material, such as a synthetic polymer. Suitable synthetic polymers include, but are not limited to, poly lactic-co-glycolic acid (PLGA), polylactic acid, polycaprolactone (PCL), nanosilicates, polyvinyl alcohol, poly(n-isopropylacrylamide), or a combination thereof. In some embodiments, the matrix comprises, consists essentially of, or consists of, a synthetic material.

The matrix may comprise one component, such as a hydrogel or synthetic material. However, in certain embodiments, such as certain embodiments where the composition form a mesh structure, the matrix may comprise two or more components, such as two or more hydrogel and/or synthetic material components. In some embodiments of a mesh composition, the matrix may comprise a first fiber comprising a first matrix component and a second fiber comprising a second matrix component, different from the first matrix component. And the mesh composition may further comprise additional matrix components, such as 1, 2, 3, or more additional matrix components, that are different from both the first and second matrix components, and from each other. In some embodiments, the first and second matrix components comprise a first and a second hydrogel, respectively.

In any embodiments, the matrix may further comprise one or more additional materials. The additional material may be any material selected to produce a desired and/or beneficial result. Exemplary additional materials that may be included in the first component include, but are not limited to, nanocomposites, such as nanoparticles of gold, silver, carbon, graphene, graphene oxide, reduced graphene oxide, zinc, Fe₃O₄ magnetic nanoparticles, or a combination thereof; conductive polymers, such as poly(3,4-ethylenedioxythiophene) polystyrene sulfonate (PEDOT:PSS); or any combination thereof.

The matrix may further comprise a photo initiator and/or a crosslinking agent. Suitable cross-linking agents and/or photo initiators include any photo initiator that facilitates cross linking of matrix threads to form a mesh. Suitable cross-linking agents and/or photo initiators include, but are not limited to, calcium chloride, 2-hydroxy-4′-(2-hydroxyethoxy)-2-methylpropiophenone, 2,2′-azobis[2-methyl-N-(2-hydroxyethyl)propionamide azo (VA-086), lithium phenyl-2,4,6-trimethylbenzoylphosphinate (LAP), Eosin Y or a combination thereof.

In some embodiments, the matrix forms a sheet or a mesh. In some embodiments, the matrix comprises plural fibers forming a mesh. Each matrix fiber has a diameter suitable for forming a mesh. The fiber diameter may be from greater than zero to 2000 μm or more, such as from 25 μm to 1500 μm, from 50 μm to 1250 μm, from 50 μm to 1000 μm, from 100 μm to 1000 μm or from 150 μm to 500 μm. In certain embodiments, the fiber has a diameter of from 150 μm to 300 μm, but in other embodiments, the finer has a diameter of from 500 μm to 1250 μm.

The fibers in a mesh composition may define pores between the fibers. These mesh pores typically are open from one side of the mesh to the other side. The pores may be of any size suitable for use in the mesh. In some embodiments, the pores may have a diameter of from greater than zero to 10,000 μm or more, such as from 50 μm to 5,000 μm, from 100 μm to 5,000 μm, or from 100 μm to 1,000 μm. The mesh may be 3D printed to any size that is required for the application. In particular embodiments, the mesh may be substantially rectangular and may have one or both sides of from 5 mm to 25 mm or more. In certain embodiments, the mesh is substantially square and may be a square having dimensions of from 5 mm×5 mm to 25 mm×25 mm or more. In other embodiments, the mesh may be substantially circular, and may have a diameter of from 5 mm to 25 mm or more.

B. Microparticle

The composition also comprises a plurality of microparticles. A microparticle typically has at least one dimension, such as one, two or three dimensions, of from 1 μm to less than 1000 μm, such as from 1 μm to 750 μm, from 1 μm to 500 μm, from 1 μm to 250 μm, from 1 μm to 100 from 1 μM to 50 or from 1 μM to 25 In some embodiments, the dimension is a microparticle diameter. And/or the microparticles may be any suitable shape, such as a microspheres or rod shape. The microparticles may be dispersed into the matrix. Typically, the microparticles comprise a material different from the material of the matrix into which they are dispersed. A microparticle may be a solid microparticle, or it may be a hollow microparticle. Materials suitable for forming a microparticle include any material that will form a microparticle that comprises a therapeutic agent. Typically, the therapeutic agent is encapsulated within the microparticle. In some embodiments, the microparticle is a biodegradable microparticle, but in other embodiments, the microparticle is not a biodegradable microparticle. Suitable microparticle materials include, but are not limited to, hydrogels, such as alginate, gelatin, agarose, hyaluronic acid, gelatin methacryloyl, chitosan, elastin, collage, polyethylene glycol, or a combination thereof; synthetic materials, such as poly lactic-co-glycolic acid (PLGA), polylactic acid, polycaprolactone (PCL), nanosilicates, polyvinyl alcohol, poly(n-isopropylacrylamide), or a combination thereof; or any combination thereof. In certain working embodiments, the microparticle comprises PLGA, PCL, or a combination thereof.

In some embodiments, the microparticle material is selected to provide an environment suitable for the therapeutic agent, and/or to protect the therapeutic agent from an environment into which the composition is placed. For example, a therapeutic agent may be adversely affected by exposure to water, certain pH levels, and/or certain enzymes, particularly over the course of a sustained release period, such as several days. In some embodiment, the microparticle is selected to provide an environment suitable to substantially stabilize the therapeutic agent, and/or reduce or substantially prevent exposure to conditions that might degrade the therapeutic agent, such as pH changes, moisture or enzymes.

Additionally, or alternatively, the microparticle may be selected to release the therapeutic agent at a desired time and/or rate. For example, the microparticle may be selected to provide a sustained release or a delayed release of the therapeutic agent. A sustained release period may be from 1 hour to 30 days or more, such as from 1 day to 30 days, from 1 day to 25 days, or from 1 day to 10 days.

The microparticle may further comprise one or more additional materials. The additional material may be any material selected to produce a desired and/or beneficial result. Exemplary additional materials that may be included in the first component include, but are not limited to, nanocomposites, such as nanoparticles of gold, silver, carbon, graphene, graphene oxide, reduced graphene oxide, zinc, Fe₃O₄ magnetic nanoparticles, or a combination thereof; conductive polymers, such as poly(3,4-ethylenedioxythiophene) polystyrene sulfonate (PEDOT:PSS); or any combination thereof.

In some embodiments, the composition comprises two or more different microparticles, such as 2, 3, 4, 5, or more different microparticles. Two different microparticles might be formed from different materials and/or might contain different therapeutic agents. That is, the plurality of microparticles might comprise a first microparticle and a second microparticle different from the first microparticle, and may further comprise a third and subsequent microparticle different from the first and second microparticles.

In certain embodiments, the composition comprises a mesh comprising at least a first matrix component comprising a first microparticle, and a second matrix component comprising a second microparticle different from the first microparticle. In such embodiments, while the microparticles are different, such as by being formed from different materials and/or containing different therapeutic agents, the first and second matrix components might be the same, or they might be different.

C. Therapeutic Agent

The disclosed composition also comprises a therapeutic agent. The therapeutic agent may be any therapeutic agent suitable for use in the disclosed composition. In some embodiments, the therapeutic agent is an anticancer drug, antiviral, antibiotic, antifungal agent, anti-parasitic, anti-inflammatory agent, pain killer, growth factor, antibody, peptide, cytokine, chemokine, immunomodulatory agent, radioactive composition, clotting aid, or a combination thereof. In certain embodiments, the therapeutic is an anticancer drug, and in particular embodiments, the anticancer drug is temozolomide; all-trans retinoic acid; doxorubicin; paclitaxel; 5-FU (5-fluorouracil, such as fluorouracil injection); doxurubicin hydrochloride; docetaxel; epirubicin; cisplatin; carboplatin; simvastatin; antibodies, such as bevacizumab, ranibizumab, and/or aflibercept; chemoattractants, including, but not limited to, bradikinin, epidermal growth factor, vascular growth factor, and/or interleukin 8; or any combination thereof.

In some embodiments, the composition comprises one therapeutic agent, but in other embodiments, the composition comprises two or more therapeutic agents, such as 2, 3, 4, 5, 6 or more therapeutic agents. A therapeutic agent may be encapsulated in a microparticle or conjugated to the matrix. Typically, the composition comprises one therapeutic agent per microparticle, but in some embodiments, a microparticle may comprise two or more therapeutic agents.

In some embodiments, the composition comprises all-trans retinoic acid encapsulated in a PCL microparticle, bevacizumab in a PLGA microparticle, ranibizumab in a PLGA microparticle, or bradykinin in a PLGA microparticle, and in certain working embodiments, the composition comprises temozolomide encapsulated in a PLGA microparticle. In some embodiments, the composition comprises a combination of these therapeutic agent/microparticle combinations, such as 2, 3, 4, or all 5 of these therapeutic agent/microparticle combinations.

### III. Method of Making the Composition

Also disclosed here are embodiments of a method for making the composition. The method may comprise encapsulating the therapeutic agent in microparticles, dispersing the microparticles within the matrix to form the composition. The method may further comprise forming the composition into a sheet or mesh, such as by 3-D printing.

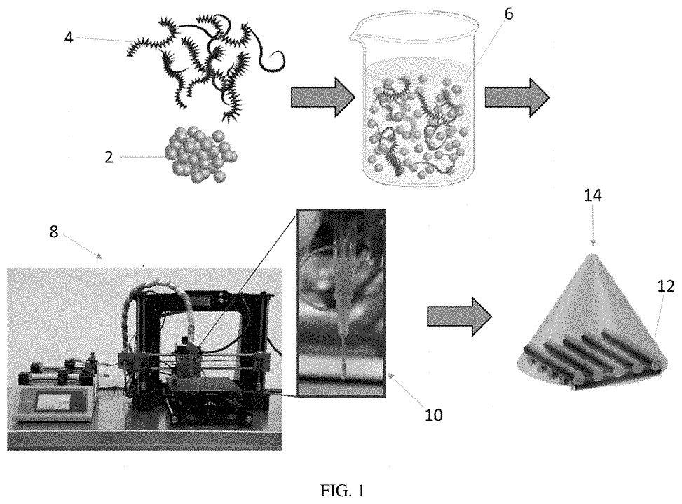

FIG. 1 provides a schematic diagram illustrating exemplary embodiments of a method for making the composition. With respect to FIG. 1, therapeutic agent-loaded microparticles 2 and combined with the matrix 4 to from a bioink 6 that is suitable for 3-D printing. Solvents suitable for use in the bioink include any solvent that facilitates 3-D printing of the matrix, such as water; alcohol, such as methanol, ethanol, propanol, iso-propanol; or a combination thereof.

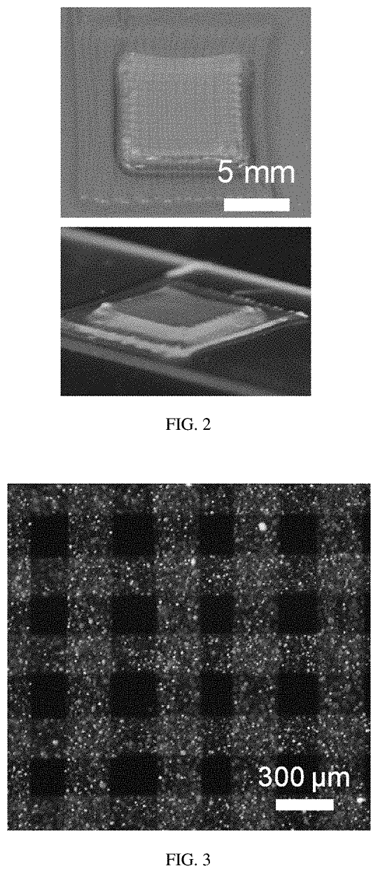

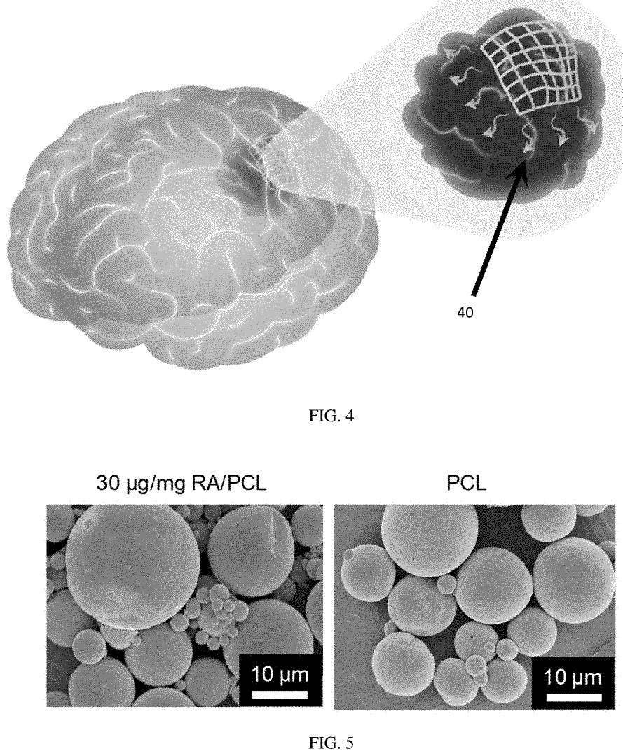

The composition is printed on a 3-D printer 8 by extruding the composition through a coaxial extruder 10 to from fibers 12. The bioink may be extruded through a single nozzle, or multiple nozzles. The fibers 12 may then be cross-linked by exposure to UV radiation 14. Alternatively, or additionally, the cross-linking may be achieved by exposure to calcium ions and/or a temperature change. FIGS. 2 and 3 provide photographic and microscope images of an exemplary composition formed as a mesh, and FIG. 4 provides a schematic diagram illustrating how the flexible mesh may be implanted into a brain and be contoured to maximize contact with the target site. And the arrows 40 in FIG. 4, represent release of the therapeutic agent into the brain.

The microparticles, optionally loaded with the therapeutic agent, may be made by any suitable method known to persons of ordinary skill in the art, such as water-in-oil, water-in-oil-in-water, oil-in-oil emulsion techniques using either batch emulsion systems or microfluidic droplet generators. In certain embodiments, the oil-in-oil emulsions are used.

### IV. Applications

The disclosed composition are useful for any application that benefits from delivery of a therapeutic agent. In some embodiments, the composition is useful to deliver a therapeutic agent to a specific location, such as a cancer tumor or a wound. In such embodiments, the therapeutic agent may be within the microparticles and the matrix may prevent the microparticles, and thereby the therapeutic agent, from moving, or being moved, away from the target site. In ether embodiments, the composition provides delayed and/or sustained release of a therapeutic agent. In such embodiments, the microparticles and/or the matrix slow down or delay the therapeutic agent from being released. Also, in embodiments where the composition comprises two or more therapeutic agents, the microparticles and/or matrix, or combination of matrices, may provide sequential delivery of the therapeutic agents.

In some embodiments, the therapeutic agent is or comprises an anticancer agent, and the composition is useful for treating cancer. In some embodiments, the cancer is a solid tumor cancer and may be a breast, lung, prostate, colon, brain, uterus, pancreas, skin, or liver cancer. In particular embodiments, the cancer is a brain cancer, such as glioblastoma multiforme. The composition may be placed, such as implanted, adjacent to or onto, a cancer tumor or other cancer cells. In such embodiments, the composition releases the therapeutic agent, optionally, over a desired time period, in a site-specific manner. In particular embodiments, the cancer is a brain cancer, such as glioblastoma multiforme, breast cancer or skin cancer. In some embodiments the cancer is Glioblastoma and the therapeutic agent is temozolomide, bevacizumab, and/or simvastatin. Alternatively, the cancer may be breast cancer and the therapeutic agent may be 5-fluorouracil, doxurubicin hydrochloride, docetaxel, epirubicin, cisplatin, and/or carboplatin. Or the cancer may be skin cancer and the therapeutic agent may be cisplatin, doxorubicin, 5-fluorouracil, capecitabine, topotecan, and/or etoposide. In some embodiments, the composition may be biodegradable, i.e. the composition may comprise a biodegradable matrix and biodegradable microparticles, such that the composition does not have to be removed, thereby eliminating the need for follow-up surgery.

In other particular embodiments, the composition is useful for treating wounds. In such embodiments, the composition may comprise a growth factor, an antibiotic agent, a pain killer, a clotting agent, an anti-inflammatory agent, or a combination thereof. The composition may be placed directly onto a wound to target delivery of the therapeutic agent(s) to the wound. The composition may be a sheet and may provide protection to the wound, such as preventing dirt and infection from accessing the open wound. Alternatively, the composition may be a mesh, and as such may facilitate tissue growth through the fibers. In either embodiments, the composition may be biodegradable, i.e. the composition may comprise a biodegradable matrix and biodegradable microparticles, such that the composition does not have to be removed as the wound heals.

### V. Examples

### Example 1

Hydrogel Mesh Loaded with all-Trans Retinoic Acid for Treatment of Glioblastoma Multiforme

### I. Overview

Glioblastoma multiforme (GBM) is a rapid progressive and deadly form of glial tumors with a median survival rate of about 15 months. The incidence of GBM is 2 to 3 per 100,000 people in North America, which accounts for 12 to 15% of all intracranial tumors and 50 to 60% of astrocytic tumors. GBMs are hard to treat and significantly affect the patient's physical and cognitive abilities and quality of life as the tumors are mostly located at the control center for thought, emotion, and movement. Moreover, GBMs place a significant economic burden on the patient, his/her family, and the healthcare system. The current standard of care for newly diagnosed GBM is the surgical resection to the extent feasible followed by 6 weeks of radiotherapy with concurrent temozolomide treatment. After the radiotherapy is finished, the monthly administration of temozolomide is maintained for 6 months up to 1 year. Together, these typically add only months of additional survival. A major challenge associated with the management of GBM is the resistance of GBM cancer stem cells to temozolomide via tightly regulated apoptosis, autophagy and unfolded protein responses. Furthermore, temozolomide has serious side effects that significantly affect the quality of life of GBM patients. Therefore, there is a pressing need for the development of novel therapeutics for the treatment of GBM with the goal of increasing the survival rate and improving the quality of the patient's life.

ATRA is a derivative of vitamin A that has been used in the chemoprevention and differentiation therapy of acute promyelocytic leukemia. Several studies have shown that ATRA can effectively inhibit the proliferation of GBM and induce apoptosis in these cells. The phosphorylation and inactivation of Bcl-2, an antiapoptotic protein, mediated by ATRA results in favorable therapeutic effects toward GBM. There is also evidence showing that ATRA may promote the effect of suicide-gene therapy against medulloblastoma, and may significantly enhance its antitumor effect on glioma when used in combination with specific chemoimmunotherapeutic agents. Altogether, these results indicate the therapeutic potential of ATRA for patients with glioma.

Oral administration is the main route of delivering ATRA. However, the short half-life of the drug (about 1.5 hours) hampers achieving efficacious drug levels in the tumor and necessitates the use of higher doses of the drug at the risk of damaging vital organs such as the liver and kidney. Depending on the hydrophobicity and hydrophilicity of the drug, single- or double-emulsion systems have been used to produce these drug-loaded microparticles. However, challenges associated with the rapid drug release, low encapsulation efficiency and the fact that these particles dislocate after implantation lead to limited application in the management of GBM.

One embodiments of the disclosed composition has been tested as a drug-eluting hydrogel mesh capable of sustained release of ATRA for over three weeks. Microextrusion bioprinting technology was used to fabricate 3D porous hydrogel meshes for local delivery of ATRA. Hydrogels were composed of alginate and GelMA biopolymers and were generated via a two-step ionic and photo-crosslinking process. Using drug-eluting meshes offers several advantages: 1) it provides large pore sizes that promote the delivery of oxygen and nutrients to the underlying tissue and enables cancer cells to infiltrate into the mesh once they reach the mesh; 2) encapsulating ATRA-releasing microspheres in the mesh prevents them from dislocation and will keep them at the implantation site for a long period; and 3) the mesh provides the flexibility suitable to conform to the irregular structure of the tumor cavity results from surgical resection. In certain disclosed embodiments, ATRA was encapsulated into polycaprolactone (PCL) microspheres, which were suspended in biopolymer solution prior to the 3D bioprinting process. The release kinetics of ATRA from PCL microspheres and from hydrogel meshes with different morphologies were determined experimentally. Finally, the cell death effect of ATRA-loaded meshes on U-87 MG cell line was evaluated in vitro, using cell counting, PrestoBlue™ viability test, and an apoptosis assay.

### II. Materials and Methods

### ATRA-Loaded PCL Microspheres Preparation

Microspheres were fabricated using an oil-water single emulsion technique. 500 mg of polycaprolactone (PCL) (Sigma-Aldrich, St. Louis, USA, Catalog No.: 704105) were dissolved in 3 ml of dichloromethane (DCM) (Anachemia, Lachine, Canada, Catalog No.: CA71007-062) and mixed on a stirring hotplate. 15 mg of all-trans retinoic acid (ATRA) (Sigma-Aldrich, St. Louis, USA, Catalog No.: R2625) were then dissolved into the solution to provide an ATRA loading of 30 μg/mg RA/PCL. While still on the stirrer, 3 ml of 100% ethanol was added to the solution. Then, 3 ml of 2% (w/v) poly vinyl alcohol (PVA) (Sigma-Aldrich, St. Louis, USA, Catalog No.: 363170) aqueous solution was slowly added while making sure not to disrupt the boundary layer. The solution was emulsified on a vortex mixer for 12 seconds and quickly added to 100 ml of 0.3% (w/v) PVA solution at 35° C. To evaporate the organic solvent, the solution was stirred for 4 hours at 500 rpm at the same temperature. After the evaporation step, microspheres were collected by 5 minutes of centrifugation at 4000 rpm and washed with 3×50 ml of dH₂O, to remove any remaining PVA. The resulting microspheres were frozen and lyophilized for 24-36 hours, and stored at −20° C. until use.

### Characterization of ATRA-Loaded PCL Microspheres

Scanning electron microscopy (SEM) was used to characterize the size distribution of the microspheres. 1 mg of microspheres was suspended in anhydrous ethanol and deposited on a SEM stub. After the ethanol evaporated, the microspheres were coated with gold-palladium (Anatech Hummer VI sputter coater). SEM imaging was conducted with 1.0 kV using a Hitachi S4800 electron microscope. Microspheres' diameter was determined based on image processing using ImageJ software.

### Determination of ATRA Encapsulation Efficiency

10 mg of the ATRA-loaded microspheres were dissolved in 300 μl of DCM. PCL was then precipitated out of the solution by adding 1.2 ml of 95% ethanol, and removed by centrifugation at 15,000 rpm. Using a microplate reader (Infinite M200 Pro), the content of ATRA in microspheres was determined by measuring the absorbance of the obtained solution at 354 nm and subtracting the similar value obtained from unloaded PCL microspheres. Encapsulation efficiency was calculated using the following equation.

$$EE ( \% ) = \frac{RA detected in microspheres}{RA used to fabricate microsphes} \times 100 \qquad Eq . 1$$

Determination of Drug Release Kinetics from Microspheres

Release studies were performed by first suspending 5 mg of ATRA-loaded microspheres in 50 ml of tris buffer (pH 7.4) (Sigma-Aldrich, St Louis, USA Catalog No.: T6066) at 37° C. At each time point, the amount of released ATRA was evaluated by determining the concentration of ATRA in the release medium via plate reading at 354 nm. The release medium was replaced in each time point with fresh tris buffer to maintain a perfect sink condition.

### Bioink Preparation

Alginate-GelMA-photoinitiator (PI) solution with suspended ATRA microspheres was used as 3D bioprinting material. For preparation of this bioink, photo initiator (2-Hydroxy-4′-(2-hydroxyethoxy)-2-methylpropiophenone, Sigma-Aldrich, St. Louis, USA, Catalog No.: 410896) was dissolved in deionized (DI) water in a concentration of 0.5% (w/v) at 60° C. by periodic vortex-mixing. Next, GelMA was added to the solution and kept at the same temperature for 15 minutes to dissolve. Alginate (Sigma-Aldrich, St. Louis, USA, Catalog No.: W201502) was then added to the solution at 60° C. while vortexing at 300 rpm to reach a desired concentration of 2% (w/v) or 4% (v/w). The mixture was stirred at 60° C. for 1 hour to have a homogeneous solution. Next, the previously prepared ATRA-loaded microspheres were suspended in alginate solution and vortex-mixed at 3000 rpm before being used for the bioprinting process.

### 3D Bioprinting and Fabrication of the 3D Bioprinter's Extruder

A microextrusion 3D bioprinter was developed by incorporating a heated coaxial core-sheath extruder and two syringe pumps into a commercial 3D printer (Prusa i3). The coaxial core-sheath extruder was used to flow the bioink and a crosslinking agent (6% (w/v) CaCl₂, Bio Basic, Markham, Canada, Catalog No.: CT1330 solution) simultaneously in the core and sheath channels, respectively, and ionically crosslink the bioink at the tip of the extruder during the printing process.

Bioink and crosslinking agent were loaded into 3-ml syringes that were attached to two syringe pumps (Harvard Apparatus, USA). Bioink was supplied with flow rates ranging from 6 μl/min to 18 μl/min and printing speeds ranging from 5 mm/s to 14 mm/s. The crosslinking agent was supplied with half and quarter of the flow rate of bioink with 2% (w/v) and 4% (w/v) alginate, respectively. During the 3D printing process, fibers formed from ionically-crosslinking bioink was deposited on the 3D printer's bed in two layers which were further photo-crosslinked by ultraviolet (UV) irradiation. A second crosslinking step was performed to provide structural integrity between 3D printed fibers and to construct a 3D integrated mesh.

Two hypodermic needles were attached to each other coaxially with the inner needle (25 gauge) fixed and maintained concentrically with the outer needle (18 gauge) by making small dimples on the sheath needle's sides. Flexible tubings were then used to connect the sheath and core needles to the calcium chloride solution and bioink syringes, respectively.

### Culture of U-87 MG Cells

U-87 MG cells were provided by ATCC® and cultured in Dulbecco's Modified Eagle Medium (DMEM), high glucose (Gibco™ by Life Technologies™, Carlsbad, USA, Catalog No.: 11965084) with 10% fetal bovine serum (Gibco™ by Life Technologies™, Carlsbad, USA, Catalog No.: 12483020), 1% penicillin-streptomycin (Invitrogen/Life Technologies™, Carlsbad, USA, Catalog No.: 15140122), and 4 μg/ml puromycin at 37° C. in an atmosphere of 5% CO2. Cells were counted by hemocytometer (Bright-Line™) with two-time dilution in Trypan Blue solution (Sigma-Aldrich, St Louis, USA, Catalog No.:93595).

### Cell Viability Assay

Cell viability was assessed by PrestoBlue™ (Invitrogen/Life Technologies™, Carlsbad, USA, Catalog No.: A13262) assay. In this experiment, cells were first seeded in 6-well plates in a density of 6×104 cells/well. Viability tests were conducted at 48, 72, and 96 hours by replacing the cell media with 10% PrestoBlu™ reagent and incubating at 37° C. for 1 hour. Following, 100 μl of the incubated 10% PrestoBlue™ reagent from each well of the assay plates was transferred to a new well in a 96-well plate and read by a microplate reader (Infinite M200 Pro). Finally, viability was calculated by the following equation:

$$Cell viability = \frac{OD of ATRA - trated cells}{OD of control cells} \times 100 \qquad Eq . 2$$

### Apoptosis Assay

To determine the level of apoptosis induced by ATRA apoptosis levels were detected using propidium iodide (PI) lysis buffer and flow cytometry. Non-stained cells were used as negative controls and cells treated with simvastatin (Sigma-Aldrich, St. Louis, USA Catalog No.: 56196) were used as positive control samples. 1% sodium citrate (Bio Basic, Markham, Canada, Catalog No.: CB0035) and 0.1% Triton X-100 (Sigma-Aldrich, Sigma-Aldrich, St. Louis, USA Catalog No.: X100) were added to distilled water to prepare an initial solution for PI lysis buffer. The pH of the solution was adjusted to 7.4 and stored.

Prior to the apoptosis assay, RNase (Sigma-Aldrich, St. Louis, USA Catalog No.: R6513) and propidium iodide (Sigma-Aldrich, St. Louis, USA Catalog No.: P4864) was added to the initial solution to reach concentrations of 0.5 mg/ml and 40 μg/ml for RNase and propidium iodide, respectively. The cell media was collected and the cells were detached with EDTA (Caledon Laboratory Chemicals, Georgetown, Canada, Catalog No.: 3460-1-65) solution. The cell suspension was centrifuged at 4° C. in 1500 g for 5 minutes. Following, the cell pellets were washed twice with 500 μl of DPBS (Gibco™ by Life Technologies™, Carlsbad, USA, Catalog No.: 14190250) 4° C. The pellets were then lysed and stained with 250 μl of PI lysis buffer, and kept at room temperature in the dark for 15-30 minutes. 200 μl of cells in the staining solution were then transferred into a 96-well plate and kept on ice. Finally, the red channel was read on the flow cytometer to determine the sub-G1 population.

### Statistical Analysis

Standard deviation represented the uncertainty in all data. Tukey post-hoc test was performed for experiments with two or more test groups. All statistical analyses and graphing were performed in Microsoft Excel.

### III. Results and Discussion

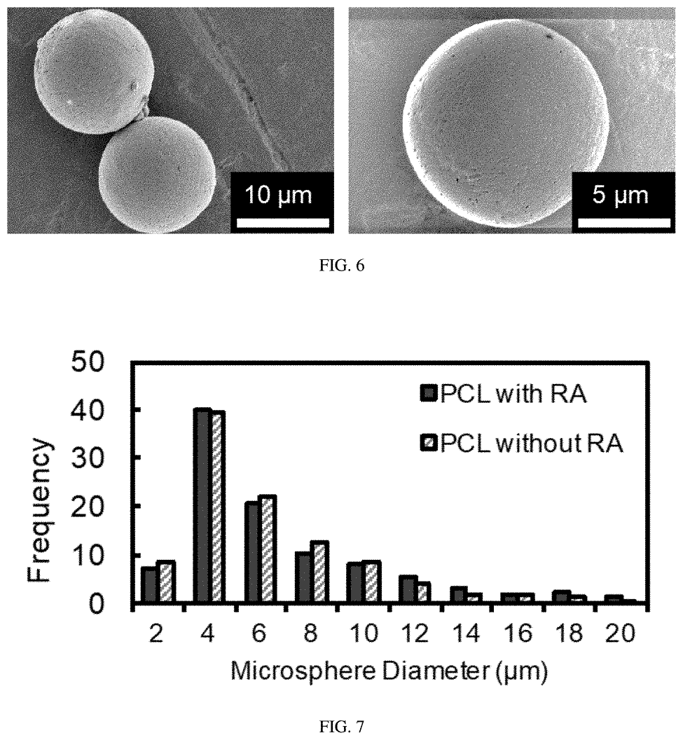

SEM images of microspheres and their size distribution are demonstrated in FIGS. 5-7. SEM images showed that the fabricated ATRA-loaded PCL particles had spherical morphology with an average diameter of 5.60±3.95 μm. Blank (unloaded) PCL microspheres, with similar morphology, had slightly smaller diameters compared to ATRA-loaded microspheres, averaging 5.15±3.40 μm.

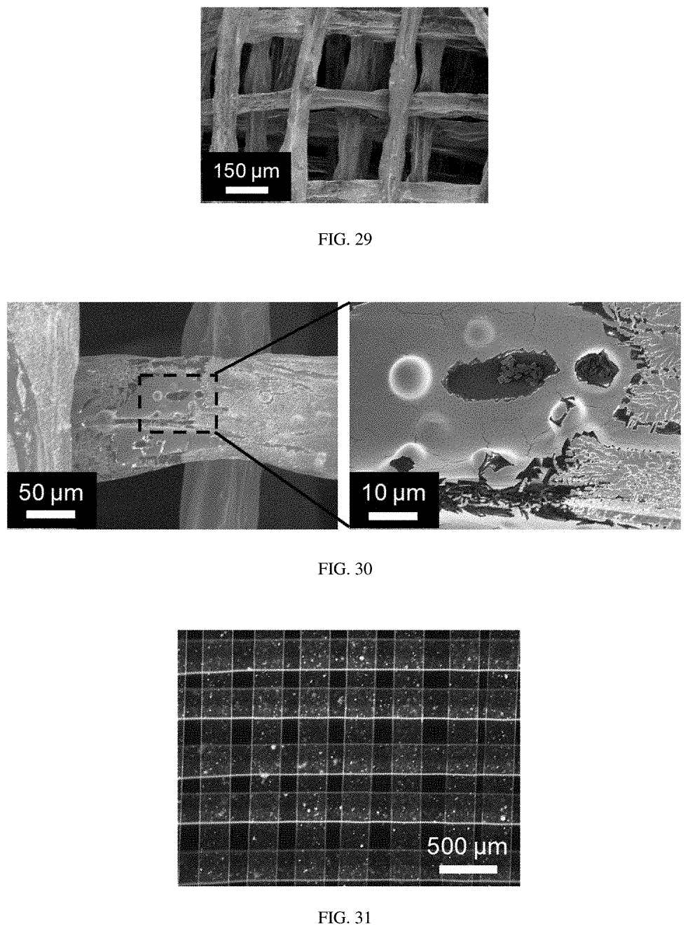

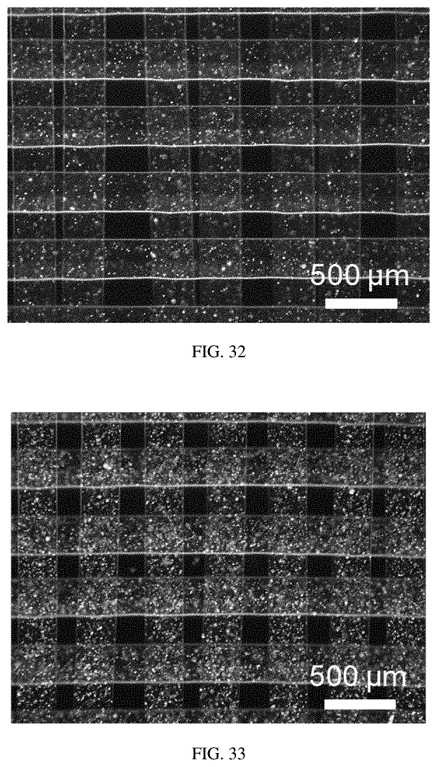

SEM images of a hydrogel mesh loaded with PCL microspheres are shown in FIGS. 29 and 30. FIG. 30 provides SEM images at increased magnification that clearly shows the presence of the microspheres in the hydrogel fiber. FIGS. 31-33 are fluorescent microscopy images of hydrogel meshes comprising different concentrations of ATRA-loaded PCL microspheres. FIGS. 31-33 clearly show a substantially uniform dispersion of microspheres inside the hydrogel structures at all the tested concentrations.

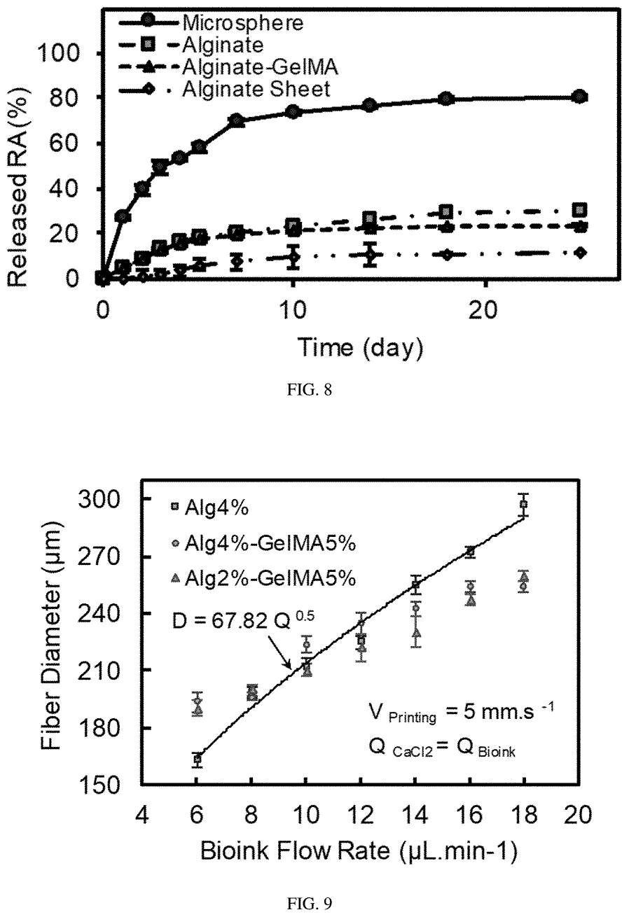

Drug release studies conducted on microspheres alone and microspheres suspended in different hydrogels, revealed that ATRA diffuses to the release medium in a faster rate when encapsulated in a microsphere alone than when the microsphere is dispersed within a hydrogel. This study of microspheres suspended in the release medium showed that after 7 days, 70% of the loaded ATRA dispersed to the medium and the release rate reduced significantly afterward, compared to the earlier stages of the drug release (FIG. 8). Similar release studies with hydrogel fibers laden with ATRA-loaded microspheres, suspended into the release medium, demonstrated a more sustained release, reaching 30.0% and 23.5% after 25 days for alginate and alginate-GelMA hydrogel fibers, respectively (FIG. 8). In addition to the hydrogel fibers, a release study was also conducted on gel sheets laden with ATRA-loaded microspheres. Possibly due to a lower surface-area-to-volume ratio, gel sheets showed a reduced release rate compared to hydrogel fibers, accumulating to 11.7% after 25 days of experiment (FIG. 8). Without being bound to a particular theory, the lower amount of released drug from hydrogels laden with microspheres, compared to the condition of microspheres suspended into the release medium, might be due to the presence of an extra barrier of hydrogel which reduces the ATRA diffusion rate.

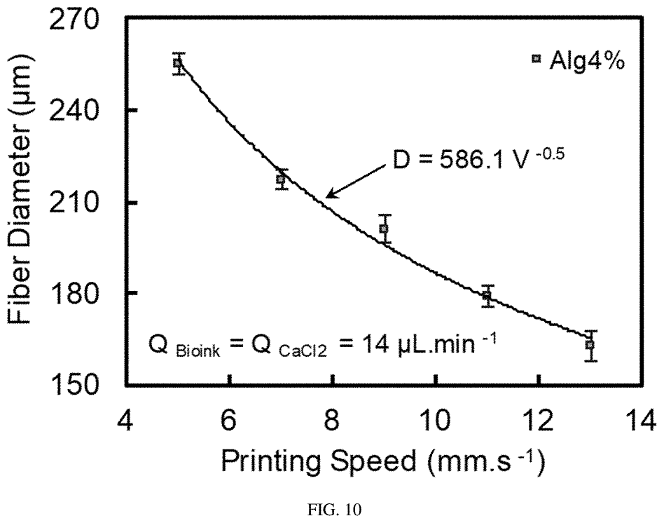

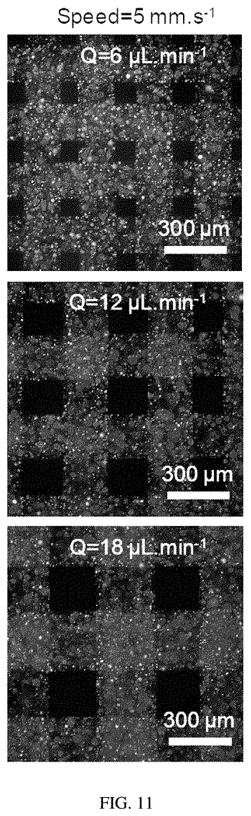

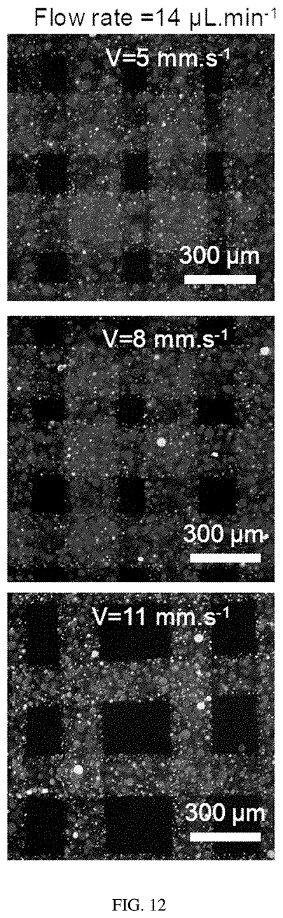

3D printed meshes were characterized by determining the effect of bioink flow rate and printing speed on the printed fibers' diameter. Bioink flow against fiber diameter was examined using three different hydrogel samples consisting of alginate 4%, alginate 4%-GelMA 5%, and alginate 2%-GelMA 5%. As indicated in FIG. 9, with bioink flow rates from 6 to 18 μl·min⁻¹, use of alginate 4% led to fiber diameters ranging from 160 μm to 300 where alginate 4%-GelMA 5% and alginate 2%-GelMA 5% resulted in fiber diameters of about 190-260 μm. Furthermore, as the printing speed increased from 5 mm·s⁻¹ to 13 mm·s⁻¹, the fiber diameter decreased from 255 μm to 163 μm (FIG. 10). FIGS. 11 and 12 provide digital images illustrating that increasing the bioink flow rate increases fiber diameter (FIG. 11), whereas printing at higher speeds results in decreasing fiber diameters (FIG. 12). With respect to FIGS. 9 and 10, each data point represents average ±SD with n=3.

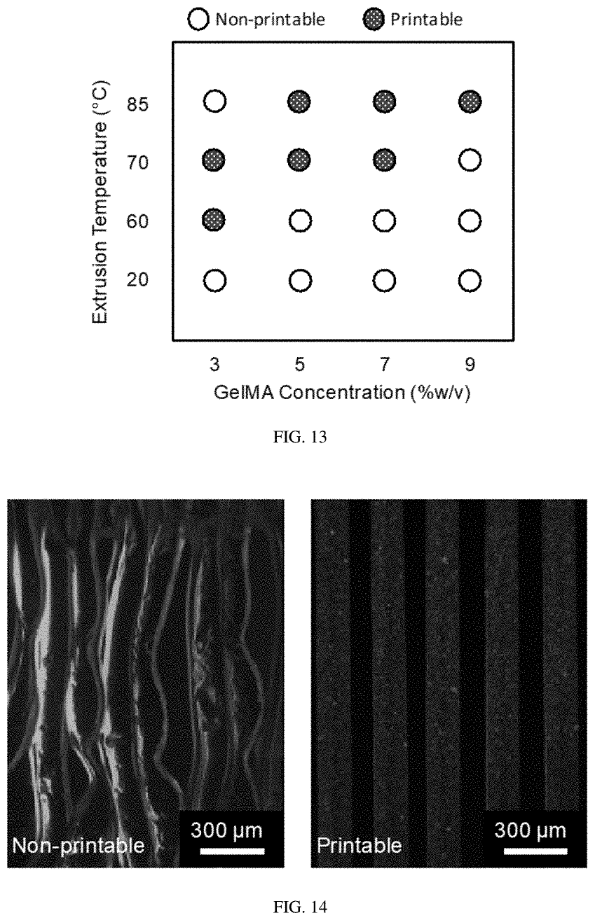

In case intermolecular interactions between gelatin and alginate affected the 3D bioprinting, a heating system was used to reduce the viscosity of alginate-GelMA blends during the 3D bioprinting process. This heating system facilitated a steady state flow of bioink in the microextrusion system and enhanced the printability of alginate-GelMA bioinks. To assess the printability of different bioinks with a constant concentration of alginate (2% w/v) and variable concentration of GelMA from 3% (w/v) to 9% (w/v), a 3D printing process was performed with different temperatures ranging from 20° C. to 85° C. Based on the results obtained, higher extrusion temperatures for higher concentrations of GelMA led to more successful printing. Typically, alginate-GelMA 3% (v/w) was printable in 60-70° C., whereas printing alginate-GelMA 9% (w/v) at below 85° C. was problematic. As indicated in FIG. 13, in this test, a combination of temperatures and GelMA concentrations yielded printable and non-printable regions with the non-printable regions displaying a non-uniform fluid flow and consequently non-uniform hydrogel fiber diameter. Microscope images of fibers prepared under printable and non-printable conditions are shown in FIG. 14.

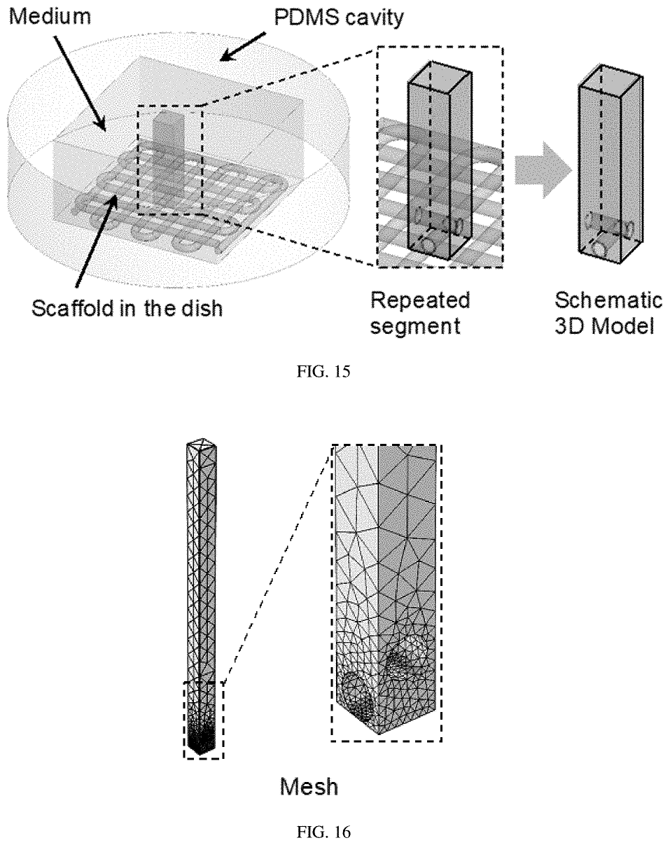

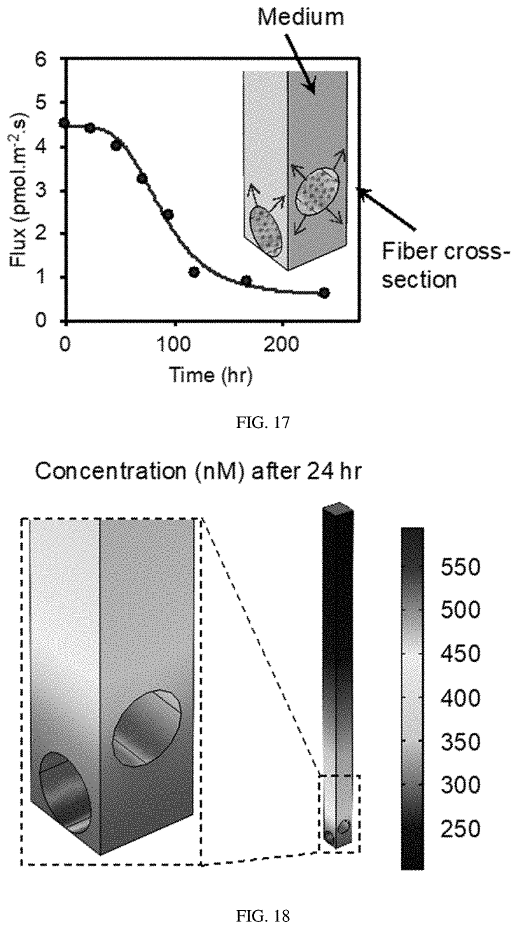

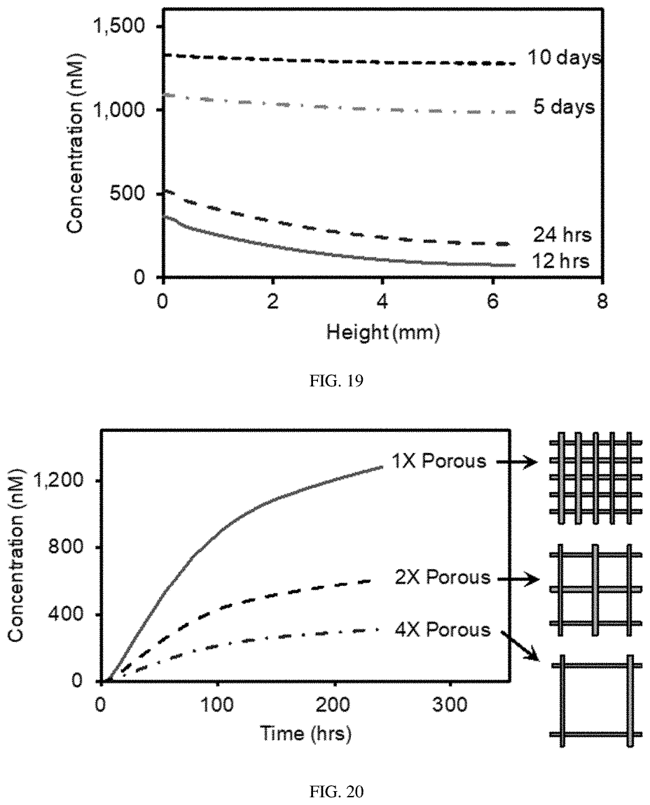

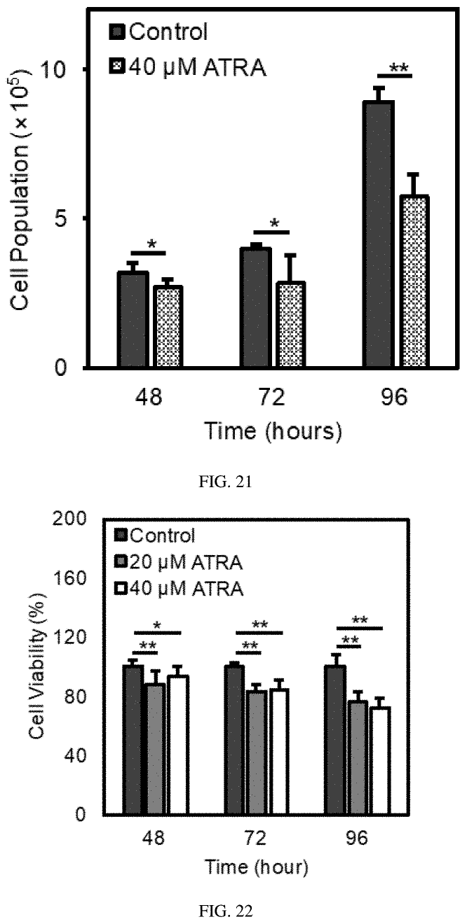

Numerical simulations were performed to study the effect of mesh porosity on the release profile of ATRA. FIGS. 15 and 16 show the numerical domain and the corresponding mesh, respectively, under the assumption that the concentration of the drug only varied in the vertical direction. Drug release was determined by solving the transient Fick's law under no-flow condition and with boundary conditions (ATRA release flux) obtained from the experimental release data (FIG. 17). The results suggested that the release rate of ATRA decreased drastically from around 20 hours to 100 hours and subtly decreased from around 100 hours to 240 hours (FIG. 17). At the beginning of drug release (12- and 24-hour time points) there was a considerable concentration gradient of ATRA in the height of the medium (FIGS. 18 and 19). This concentration gradient decreases through time as an almost uniform concentration was observable in the 10-day time point, as demonstrated in FIG. 20. Meshes with different porosities were modeled in the developed simulation platform to evaluate the effect of fiber spacing in the fabrication of hydrogel mesh on the drug release behavior. As shown in FIG. 21, by increasing the fiber spacing with 2- and 4-time factors, different release profiles were achieved, representing the capability of 3D bioprinting method to fabricate hydrogel meshes with tunable drug release behavior.

A cell count assay was performed to determine the effect of free ATRA in the cell medium on the proliferation of U-87 MG cells. As shown in FIG. 21, the presence of 40 μM of ATRA reduced the cell populations to 86.0%±7.2% and 64.4%±8.6% of the cell population in control condition, after 48 and 96 hours of culture, respectively. The effect of ATRA on the viability and proliferation of U-87 cells was further assessed by performing PrestoBlue™ assays. In these experiments, cells were exposed to ATRA in different forms including free ATRA in the cell medium, hydrogel sheets laden with ATRA-loaded PCL microspheres, and hydrogel meshes laden with the same type of microspheres. 20 μM of ATRA in the cell medium led to 88.4%±8.3% of viability after 48 hours and 77.3%±6.5% after 96 hours when compared to the control condition (FIG. 22). This impact was statistically significant in comparison to the control condition with P<0.005. The use of higher concentration of ATRA in the cell medium (40 μM) resulted in a lower cell viability, 72.4%±7.0% after 96 hours, calculated based on comparison with the control condition.

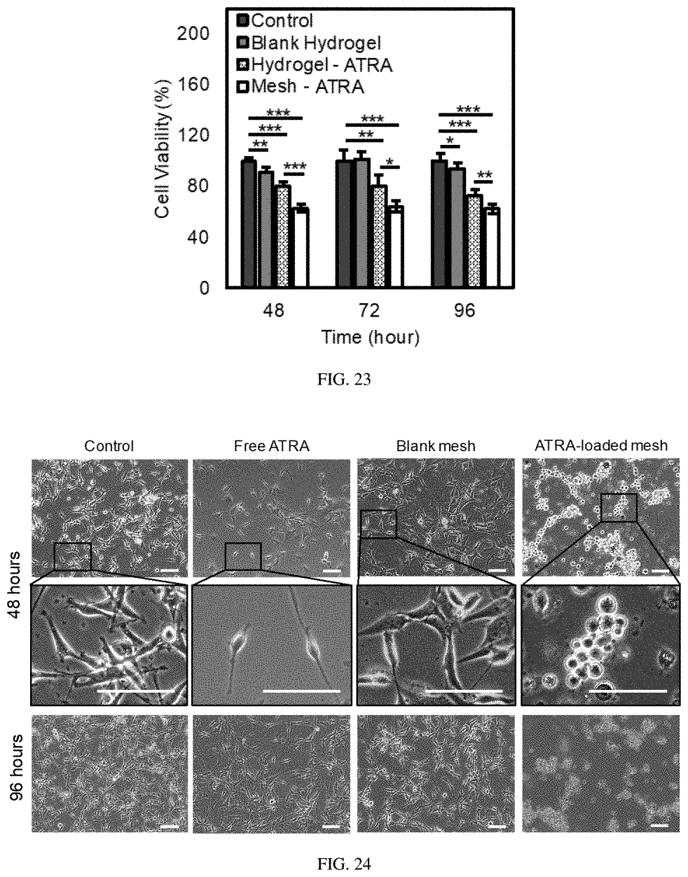

Similar tests for ATRA-loaded hydrogel sheets resulted in a cell viability of 80.5%±3.6% after 48 hours and 72.1%±5.8%, after 96 hours (FIG. 23). Cell viability was further reduced to 62.1%±3.8% after 96 hours of exposure to ATRA-loaded hydrogel meshes compared to the exposure to either ATRA directly or ATRA-loaded hydrogel sheet. Significant differences also occurred between the conditions with ATRA-loaded hydrogel sheets and ATRA-loaded meshes in different time points with P values ranging from below 0.0005 to 0.05. Without being bound to a particular theory, increased effectiveness of ATRA meshes compared to ATRA hydrogel sheets may be due to faster release of ATRA from meshes due to their higher surface-area-to-volume ratio, as previously illustrated in FIG. 8.

ATRA treatment in different forms from free ATRA to ATRA-loaded hydrogel led to lower cell confluences in the associated wells (FIG. 24). This can be interpreted as reduced viable cell population caused by ATRA. Additionally, introduction of free ATRA led to cell shrinkage, while ATRA-loaded mesh resulted in the apoptotic morphology for the majority of cells. Free ATRA represents exposure to 40 μM of ATRA. Other than ATRA, as represented in FIG. 23, blank alginate-GelMA hydrogel appeared to reduce the cell viability after 48 and 96 hours of experiment.

This effect was further confirmed by the apoptosis assay that was conducted to determine the death mechanism induced by ATRA. Similar to the viability test, this test was performed with both direct exposure to ATRA and exposure to ATRA-loaded hydrogel. Free ATRA with concentration of 40 μM was used to determine the level of apoptosis stimulated by this molecule.

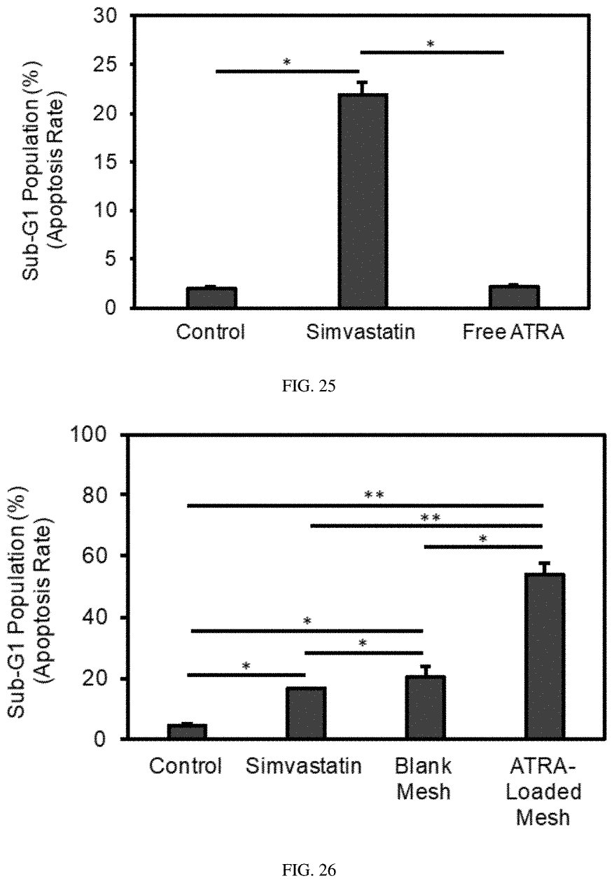

As shown in FIG. 25, direct exposure to ATRA did not result in a considerable difference in the apoptosis level when compared to the control condition, both leading to about 2% of apoptosis after 48 hours. As opposed to the free ATRA in the cell medium, hydrogel meshes laden with ATRA-loaded PCL microspheres induced a considerable level of apoptosis, as shown in FIG. 26. In this experiment, ATRA-loaded hydrogel meshes led to 53.8%±3.7% of sub-diploid population fraction which indicated the fraction of cells that underwent the apoptosis process. Treatment with ATRA-loaded hydrogels also represented significant reductions of G1 and G2 populations, confirming the previous results on the viability of U-87 MG cells when exposed to ATRA-loaded meshes. Results from samples treated with blank hydrogel mesh also showed a stimulated apoptosis level of 20.3%±4.0%. As mentioned before, these results can be compared to the reduced cell viability, resulting from exposure to the blank alginate-GelMA hydrogel. In both assays, simvastatin, an apoptosis inducer molecule, was used as a positive control condition. On average, exposure to simvastatin led 19.3%±2.8% of the cell population to undergo apoptosis in the first and second experiments.

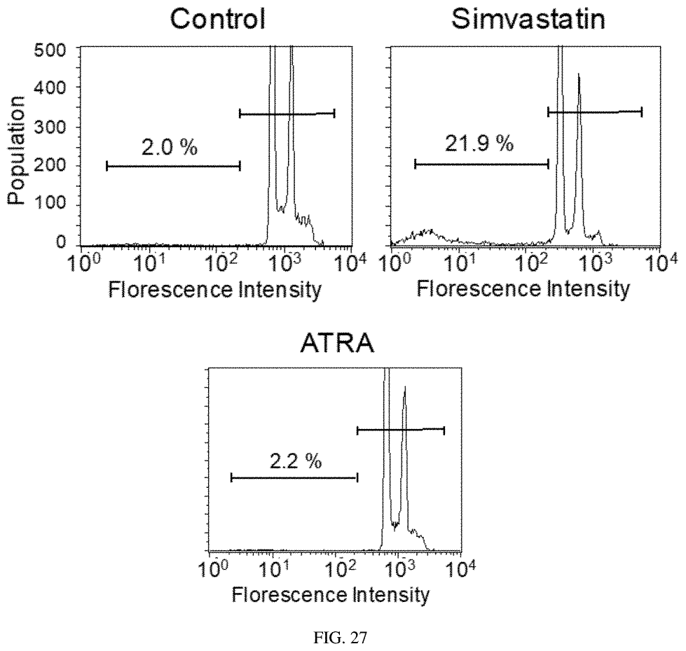

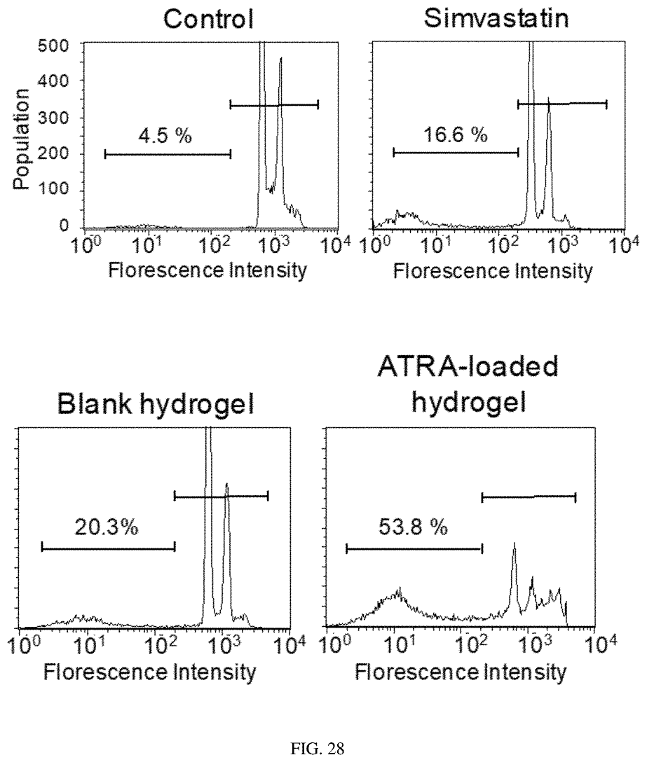

Based on the cell counting, PrestoBlue™, and apoptosis assays, exposure to free ATRA in the cell medium appeared to reduce the cellular proliferation (FIGS. 21 and 22), while not leading to any considerable apoptosis in U-87 MG cells (FIGS. 25 and 27). However, in addition to reducing the cellular proliferation, ATRA-loaded meshes significantly induced apoptosis in U-87 MG cells, which might be attributed to the sustained release of ATRA. Without being bound to a particular theory, this effect might be due to the inactivation of free ATRA via conversion to hydroxylated derivatives, in which case, encapsulation in a polymeric substrate such as PCL will promote the longevity of the drug, leading to an enhanced effectiveness toward the GBM cells.

### SUMMARY

The capability of the developed ATRA-loaded hydrogel mesh to induce apoptosis and reduce the viability of U-87 MG cells indicated a favorable therapeutic method with a less cytotoxic drug compared to other chemotherapeutic agents. Sustained release of ATRA from the hydrogel meshes facilitates a long-term therapy without a need for periodic systemic administration of the drug. Furthermore, the localized drug delivery enables the use of a lower dosage of the drug and reduced drug concentration in the serum, resulting in a reduced side-effect. The employment of alginate-GelMA hydrogel immobilizes the ATRA-loaded particles, and due to its favorable mechanical properties, accommodates an ease of use and implantation at the tumor site. Moreover, encapsulating the drug-loaded microparticles in the mesh prevents their dislocation caused by the cerebrospinal fluid.

### Example 2

### Hydrogel Mesh Loaded with Temozolomide for Treatment of Glioblastoma Multiforme

### I. Overview

Glioblastoma multiforme (GBM), classified by the World Health Organization (WHO) as a grade IV glioma, constitutes approximately half of the 21,800 patients diagnosed with primary brain tumors in the United States. Despite advances in neuro-oncology, the five-year overall survival of patients who are suffering from GBM is only 9.8%. The standard therapy for GBM is maximal safe surgical resection, followed by radiotherapy plus concomitant and adjuvant chemotherapy. Surgical intervention alone leads to a median survival of about 6 months. The median survival of patients with GBM extends to 12.1 months when surgery is accompanied with radiotherapy. Post-surgical radiotherapy alongside concomitant and adjuvant chemotherapy further prolong the median survival to 14.6 months. One reason for poor survival rate is that current treatment modalities have several deficiencies. Complete surgical resection is virtually impossible due to the diffuse nature of high-grade glioma, and even when feasible invasive surgical resection causes severe neurological insults to the surrounded healthy tissue. Presence of hypoxic regions within GBM results in resistance to radiation. Temozolomide (TMZ), an oral DNA-alkylating agent, is widely used as the adjuvant chemotherapy for GBM. Although TMZ is able to cross the blood-brain barrier (BBB), its short half-life in plasma necessitates high systemic administration doses to achieve therapeutic levels. Moreover, the prolonged oral administration of TMZ has numerous side effects including nausea, vomiting, fatigue, headache, and lymphopenia.

The recurrence of glioma at the original location of the tumor in 90% of patients necessitates the development of a localized drug delivery system capable of releasing the chemotherapeutic agent at the tumor site in a prolonged manner. Localized drug delivery systems offer the advantages of maximizing the treatment efficacy while minimizing systemic toxicity. Drug-loaded biodegradable polymer-based devices have been one of the major strategies used to achieve localized drug delivery in the clinical arena. Gliadel® wafer, a biodegradable polymer-based system loaded with carmustine (BCNU), was approved by the US Food and Drug Administration for the treatment of GBM. Similar to TMZ, BCNU is an oral DNA-alkylating agent. Eight dime-size Gliadel® wafers implanted into the tumor cavity after surgical resection are capable of releasing BCNU in a sustained manner over a 2-3-week period. Additionally, Gliadel® wafers can alleviate the adverse side effects associated with systemic administration of BCNU, including nausea, vomiting, myelosuppression, pulmonary fibrosis, hepatotoxicity, and nephrotoxicity. Despite the survival benefits of commercially available Gliadel® wafers, the size and rigid nature of these wafers make their implantation into the irregularly shaped tumor tissue difficult and complex.

Another attractive localized drug delivery strategy involves using drug-loaded poly (lactic-co-glycolic acid) (PLGA) microspheres. PLGA is a well-known biocompatible and biodegradable polymer approved by the US Food and Drug Administration for medical applications. TMZ-loaded PLGA microspheres are commonly fabricated by using oil-in-water (O/W) and water-in-oil-in-water (W/O/W) emulsion solvent evaporation methods. Previous studies reported low encapsulation efficiencies for the prepared TMZ-loaded PLGA particles with both O/W and W/O/W emulsion techniques (<7%) due to the fast diffusion of amphiphilic TMZ from the inner phase to the outer water phase during the fabrication process. Therefore, the current methods of preparation of TMZ-loaded PLGA microspheres are not cost-effective and inhibit the ability to scale-up the process.

Disclosed herein is a hydrogel-based mesh containing TMZ-loaded PLGA microspheres with high encapsulation efficiency, capable of releasing TMZ at the tumor site over a prolonged period without systemic exposure. Additionally, disclosed herein is an oil-in-oil (O/O) emulsion solvent evaporation procedure that produced TMZ-loaded PLGA microspheres with a high encapsulation efficiency. The use of liquid paraffin as the external oil phase in which TMZ is poorly soluble prevents the diffusion of the drug outwards and allows for obtaining encapsulation efficiencies as great as 61%. The aforementioned TMZ-loaded PLGA microspheres were then patterned into a drug-releasing mesh by the use of microextrusion 3-dimensional (3D) bioprinting with the aim of immobilization of the microspheres around the target site. The disclosed localized drug delivery system offers numerous advantages over current methods of GBM treatment, such as providing a sustained release of TMZ over seven weeks at the tumor site which, in turn, contributes to improving the treatment efficacy and reducing the systemic toxicity. Due to the flexibility of the disclosed hydrogel composition, it can conform to the irregular structure of brain tissue. Conformal contact then allows for high dosages and uniform distribution of TMZ. Finally, the suitable porosity provided by 3D bioprinting facilitates the transport of oxygen and nutrients to the brain tissue.

### II. Materials and Methods

Preparation of TMZ-Loaded PLGA Microspheres with O/W Single Emulsion Solvent Evaporation Method

Briefly, 0.2 g of poly (lactic-co-glycolic acid) (50:50) (PLGA) (Resomer RG504H) (Sigma, St. Louis, USA) was dissolved at room temperature in 16 mL of dichloromethane (Fisher Scientific). Afterward, 3.75 mg of temozolomide acid (Ontario Chemicals Inc., On, Canada) was dispersed into the previous solution to obtain the oil phase. The oil phase was then emulsified with 80 mL of an aqueous phase containing 2% (w/v) polyvinyl alcohol (PVA) (Sigma, St. Louis, USA) using a vortex mixer. The prepared O/W emulsion was continuously stirred at 35° C. for 4 hours to remove the organic solvent. The resulting microspheres were centrifuged and washed twice with deionized water to remove any traces of PVA and then freeze-dried. TMZ-loaded PLGA microspheres were also prepared with the saturated water phase, using the same procedure as O/W emulsion solvent evaporation method. However, in this case, the aqueous phase containing 2% (w/v) PVA was saturated with TMZ before the addition of the oil phase.

Preparation of TMZ-Loaded PLGA Microspheres with W/O/W Double Emulsion Solvent Evaporation Method

PLGA microspheres loaded with TMZ were also fabricated via a W/O/W double emulsion technique. The first aqueous phase was prepared by dissolving 3.75 mg of TMZ in 3 mL of 1% (w/v) PVA solution. This water phase was then emulsified with 10 mL of a dichloromethane solution containing 125 mg of PLGA by vortexing. Subsequently, the primary W/O emulsion was dispersed into 12.5 mL of 0.5% (w/v) PVA solution. The W/O/W emulsion was formed using a vortex mixer. Afterward, the W/O/W emulsion was poured into 60 mL of 0.2% (w/v) PVA solution and kept at 35° C. for 4 hours to allow the evaporation of the organic solvent. The final micro spheres were centrifuged and washed three times with deionized water and then freeze-dried.

Preparation of TMZ-Loaded PLGA Microspheres with Oil/Oil (O/O) Emulsion Solvent Evaporation Method

For the O/O emulsion method the first oil phase was prepared by dissolving a known amount of PLGA and 3.75 mg of TMZ into 3 mL of acetonitrile (Caledon Laboratories, Georgetown, On, Canada). 40 mL of viscous liquid paraffin (Caledon Laboratories, Georgetown, On, Canada) containing 200 μL of Span 80® (Sigma, St. Louis, USA) was used as the second oil phase. The first oil phase was then emulsified by the second oil phase using a vortex mixer. Following this, the formed emulsion was continuously stirred at 55° C. for 2 hours. The microspheres were collected by centrifugation and washed them three times with n-hexane (Fisher Scientific) in order to remove any remaining liquid paraffin and Span 80®. The resulting microspheres were air dried for 48 hours. PLGA microspheres loaded with TMZ also prepared with the saturation of acetonitrile with TMZ (30 mg) using the described O/O emulsion method.

### Determination of Encapsulation Efficiency

After accurately weighing, the microspheres that were prepared with O/W and W/O/W emulsion techniques were dissolved completely in dichloromethane via vortexing, while acetonitrile was used as the solvent for the microspheres fabricated by O/O emulsion. The supernatants were collected after multiple centrifugations at 15,000 rpm for 5 minutes. The TMZ content of each supernatant was analyzed using a plate reader (Tecan Infinite® M200Pro) at λₘₐₓ 327 nm. Equation 3 (Eq. 3) was used to calculate the encapsulation efficiency (EE) of TMZ-loaded PLGA microspheres:

$$Encapsulation efficiency ( \% ) = \frac{{D}_{m}}{{D}_{t}} \times 100 \qquad Eq . 3$$

With respect to Eq. 3, Dₘ is the actual amount of TMZ in the final microspheres and Dₜ is the initial amount of TMZ used for the fabrication of microspheres.

Characterization of PLGA Microspheres Prepared with Different PLGA Concentrations

Fabricated TMZ-loaded and blank PLGA microspheres with different polymer concentrations were characterized by scanning electron microscopy (SEM) using a Hitachi S-4800 microscope. For this purpose, 1 mg of each type of microspheres was dispersed in 1 mL of anhydrous ethanol. 10 μL of the prepared suspensions were deposited on SEM stubs and air-dried. Prepared SEM samples were sputter-coated with gold-palladium using an Anatech Hummer VI coater. SEM images were analyzed by the commercially available ImageJ software to quantify the microsphere diameter.

### Fabrication and Characterization of 3D Bioprinted Alginate Mesh

A 3D bioprinter (CELLINK AB, Gothenburg, Sweden) was used to fabricate both TMZ-releasing and blank meshes with a previously developed method. The desired amount of TMZ-loaded PLGA microspheres (based on the required TMZ content in the final mesh) was dispersed into 5 mL of deionized water. Sodium alginate (Sigma, St. Louis, USA) was then added to the previous solution to obtain a total alginate concentration of 16% (w/v). The prepared solution was vortexed periodically to obtain a homogenous suspension. The alginate solution was then loaded into the cartridge of the 3D bioprinter and printed with a speed of 400 mm/min and pressure of 80 kPa in normal condition. Alginate was first deposited on the printer's bed using a single-needle extrusion system, and furtherly crosslinked by adding 4% (w/v) CaCl₂ (Bio Basic Inc., Toronto, Canada) solution on top. Blank meshes were printed with the same procedure without adding TMZ-loaded PLGA microspheres into the alginate solution. Printed meshes with varying print head pressure, printing speed, and microsphere concentration were observed using a light microscope (Zeiss Axio Observer, Oberkochen, Germany), and the individual fiber diameters were measured using the commercially available ImageJ software.

### Fabrication of Alginate Fibers

Both TMZ-releasing and blank alginate fibers were prepared by using a wetspinning technique. Alginate solutions were prepared by the same procedure described for the fabrication of 3D bioprinted alginate meshes. A syringe pump (Harvard Apparatus) was used to pump the resulting alginate solution with an infusion rate of 500 μL min⁻¹ into a 4% (w/v) CaCl₂ coagulation bath. Ionic crosslinking of alginate fibers occurred through displacement of the divalent calcium ion with the monovalent sodium ion within the sodium alginate. Two different hypodermic needles were used, 18 and 21 gauge, to wetspin fibers with a diameter in the range of the 3D bioprinted meshes' diameters.

### In Vitro Release Studies

In vitro release studies were conducted in triplicate at 37° C. A measured amount of TMZ-loaded PLGA microspheres was dispersed in 1 mL of tris buffer (pH 6.86) (Sigma, St. Louis, USA) in an Eppendorf tube. At predetermined time intervals, samples were taken out from the incubator and centrifuged at 15,000 rpm. The entire supernatant was then collected and replaced with fresh release medium. The withdrawn supernatant was analyzed using a plate reader at λₘₐₓ 327 nm. In order to determine the release kinetics of the alginate fibers loaded with microspheres, the loaded fiber were placed into a dialysis tubing (Fisher Scientific) containing 1.5 mL of tris buffer. Dialysis tubing was then placed into a Falcon tube containing 4.5 mL of tris buffer. The Falcon tubes were kept in the incubator at 37° C. At specific time intervals, the whole medium was withdrawn from the Falcon tubes and replaced with fresh medium. A plate reader was used to determine the released amount of TMZ as described previously.

### Cell Culture

Human glioblastoma cell line, U87, was provided by ATCC®, and cultured in 25 cm² flasks (Sigma, St. Louis, USA). Cells were maintained in Dulbecco's modified Eagle medium (DMEM) (Thermo Fisher Scientific) supplemented with 10% fetal bovine serum (FBS) (Thermo Fisher Scientific), 1% penicillin/streptomycin (Life Technologies, Inc.), and 4 μg mL⁻¹ puromycin (Sigma, St. Louis, USA), and incubated at 37° C. with 95% relative humidity and 5% CO₂. The cell medium was changed every 2 days.

### Cell Viability Assay

PrestoBlue™ cell viability assay was used to determine the effect of TMZ on the viability and proliferation of U87 glioblastoma cells. In this assay, metabolically active cells reduce a non-fluorescent compound (resazurin) in the PrestoBlue™ solution into a fluorescent molecule (resorufin).

In this study, U87 glioblastoma cells were seeded in a 96 well plate at 5,000 cells per well density, and a 24 well plate at 25,000 cells per well density to evaluate the cytotoxicity of free TMZ and one embodiment of the disclosed composition, respectively. The wells were then incubated for 24 hours at 37° C. with 95% relative humidity and 5% CO₂. To determine the effect of the free TMZ, U87 glioblastoma cells were treated with different concentrations of the drug from 100 μM to 1000 μM in the cell medium. To study the effect of the disclosed hydrogel composition, the cells were treated with both microsphere-loaded (TMZ concentration of 100 μM) and blank meshes. Cells were incubated for 24, 48, 72, and 96 hours without changing the cell culture medium. At the predetermined intervals, viability assay was conducted by adding the PrestoBlue™ reagent (Thermo Fisher Scientific) at a 1:9 ratio with cell culture medium to the wells and incubating for an hour. Subsequently, the cell culture medium containing presto blue was transferred into a new well plate and the fluorescent intensity of each well was measured at excitation wavelength of 535 nm and emission wavelength of 615 nm by using a plate reader. Cell viability was then calculated by using equation 4 (Eq. 4):

$$Cell viability ( \% ) = \frac{absorbance of treated cells}{absorbance of control cells} \qquad Eq . 4$$

### III. Results and Discussion

### 3D Bioprinted Hydrogel-Based Mesh Containing TMZ-Loaded PLGA Microspheres for Glioblastoma Multiforme Management

PLGA microspheres are suitable as a TMZ localized delivery carrier due to the biocompatibility and biodegradability of the polymer. However, low encapsulation efficiencies have been reported for the prepared TMZ-loaded PLGA micro spheres with common emulsion solvent evaporation techniques including O/W, and W/O/W. These methods of fabrication are not cost-effective because a considerable amount of TMZ used in the fabrication process does not encapsulate within microspheres, possibly due to the diffusion of amphiphilic TMZ to the external phase. Therefore, a new fabrication method that produces PLGA microspheres with an improved encapsulation efficiency would be advantageous. Disclosed herein is an O/O emulsion solvent evaporation technique that fabricated PLGA microspheres with high TMZ encapsulation efficiency. Liquid paraffin was used as the outer oil phase in which TMZ has a low solubility. The poor solubility of TMZ, in turn, inhibits the drug partitioning into the outer phase. The prepared TMZ-loaded PLGA microspheres were then embedded into an alginate mesh by using a microextrusion 3D bioprinting technology. Alginate is a naturally-derived polysaccharide with broad biomedical applications including drug delivery, cell encapsulation, and tissue engineering due to its biocompatibility and ease of gelation. The incorporation of polymeric microspheres into the 3D bioprinted mesh offered the advantage of immobilizing the microspheres and achieving a prolonged TMZ release at the tumor site. Furthermore, the porosity of the 3D bioprinted construct allows the transport of oxygen and required nutrients to the brain tissue. Finally, the flexibility of the resulting hydrogel mesh allows for utilizing the entire resection cavity which then contributes to achieving high doses and uniform distribution of TMZ in the irregular contours of the brain tissue.

### Effect of Different Methods and Parameters on the Encapsulation Efficiency

Several methods and parameters were tested to improve the encapsulation of TMZ within PLGA microspheres. Table 1 provides the encapsulation efficiency of TMZ-loaded PLGA microspheres prepared with various PLGA concentrations using four different procedures. Poor encapsulation efficiencies of 0.87±0.52%, and 1.34±0.03% were observed for the microspheres prepared with O/W and W/O/W emulsions, respectively, possibly due to the rapid diffusion of amphiphilic TMZ from the inner phase into the external water phase during the evaporation of the organic solvent. In order to inhibit the diffusion of TMZ outwards, the aqueous phase was saturated with TMZ before the emulsification process in the O/W technique. However, the results did not demonstrate a considerable enhancement in the encapsulation efficiency compared to the ones prepared without saturation (5.50±1.18% as opposed to 0.87±0.52%). Without being bound to a particular theory, a significant amount of TMZ might be lost due to washing the microspheres, because the TMZ was close to the interface of the oil and aqueous phase (encapsulated near the surface of the microspheres) in this method of fabrication.

It was hypothesized that using an external oil phase in which TMZ is poorly soluble could significantly improve the encapsulation efficiency. Microspheres were fabricated with the same amount of TMZ and PLGA concentration but by using O/O emulsion technique. These O/O emulsion microspheres showed the high encapsulation efficiency of 48.30±6.20%, possibly due, at least in part, to the low solubility of TMZ in liquid paraffin that was used as the outer oil phase. Furthermore, by increasing the PLGA concentration from 1.25% to 10%, the encapsulation efficiency increased from 48.30±6.20% to 61.15±6.80%. Without being bound to a particular theory, the high viscosity of concentrated PLGA solution might be slowing down the diffusion of TMZ outwards.

TABLE 1
Encapsulation efficiency of TMZ-loaded PLGA microspheres
prepared with different techniques and parameters.

| | PLGA concentration | Encapsulation efficiency |
|---|---|---|
| Fabrication method | (w/v, %) | (%) |
| O/W | 1.25 | 0.87 ± 0.52 |
| O/W with saturation | 1.25 | 5.50 ± 1.18 |
| W/O/W | 1.25 | 1.34 ± 0.03 |
| O/O | 1.25 | 48.30 ± 6.20 |
| O/O | 5 | 58.03 ± 2.60 |
| O/O | 10 | 61.15 ± 6.80 |

Characterization of Microspheres Prepared with Different PLGA Concentrations

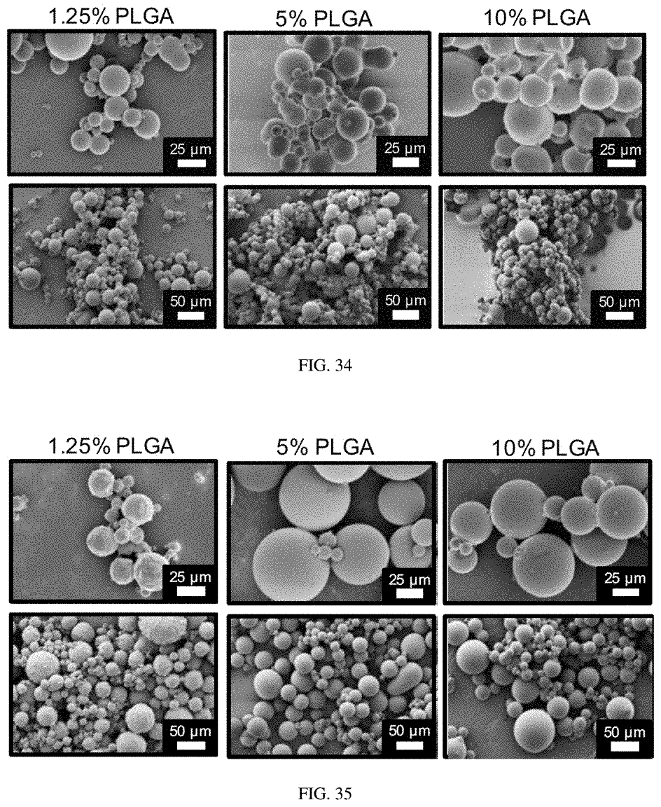

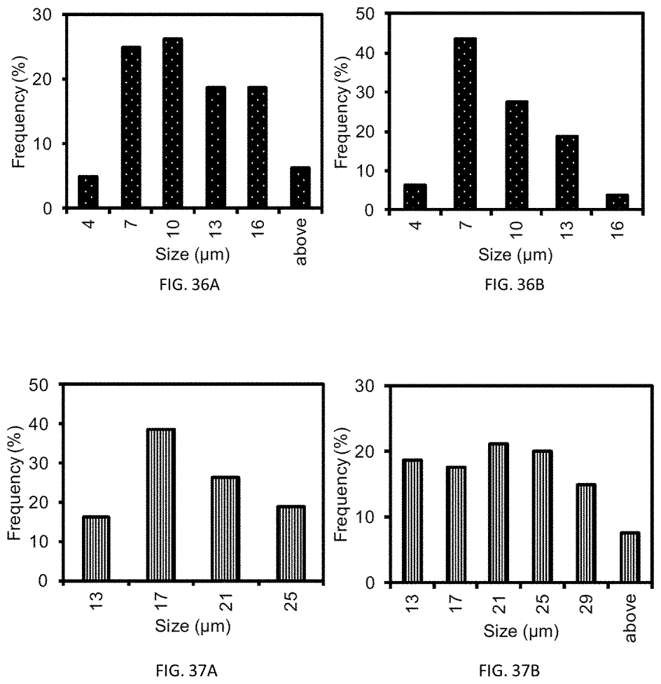

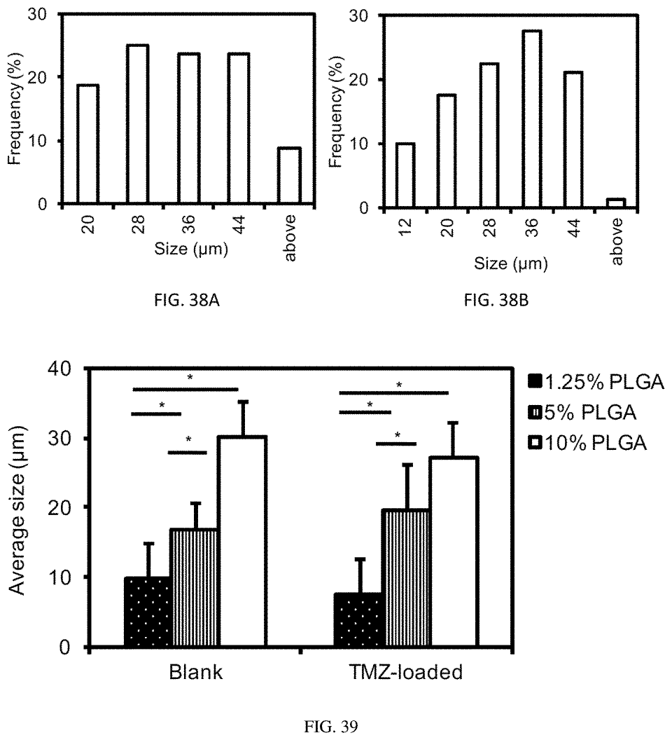

FIGS. 34 and 35 provide SEM images that show that the fabricated microspheres with different conditions had a spherical morphology. Prepared PLGA microspheres with different concentrations of PLGA were characterized based on their size (FIGS. 36A and B, 37 A and B, and 38 A and B). This characterization was performed for both blank microspheres (FIGS. 36A, 37A and 38A) and TMZ-loaded (FIGS. 36B, 37B and 38B) microspheres. With the same concentration of PLGA, no significant difference in the average size was observed between loaded and blank particles. In other words, the average size of microspheres was more dependent on the PLGA concentration than the drug loading condition. As shown in FIG. 39, the analysis of SEM images revealed that TMZ-loaded PLGA microspheres prepared with 1.25%, 5%, and 10% PLGA concentrations had an average size of 7.61±2.74 μm, 19.53±6.66 μm, and 27.15±10.04 μm, respectively. And the average size of blank PLGA microspheres prepared with 1.25%, 5%, and 10% PLGA concentration was 9.83±3.91 μm, 16.82±3.75 μm, and 30.29±9.71 μm, respectively. Without being bound to a particular theory, the PLGA concentration may have contributed to a more viscous polymer solution, which then resulted in the fabrication of larger microspheres.

### Characterization of TMZ-Releasing Alginate Mesh

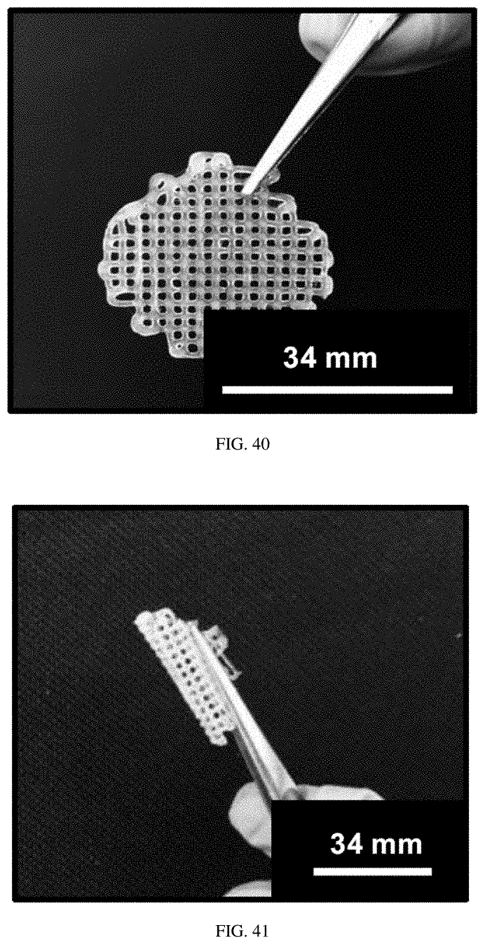

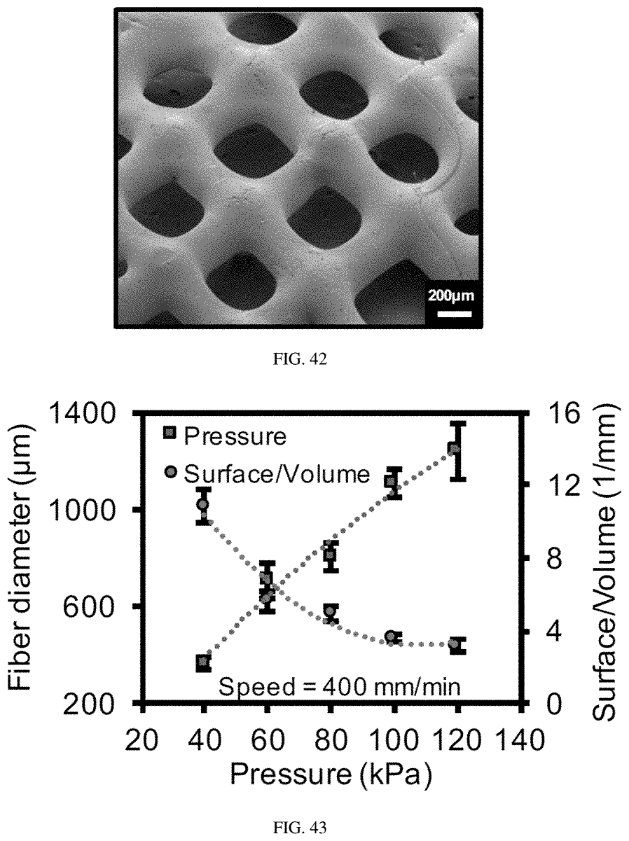

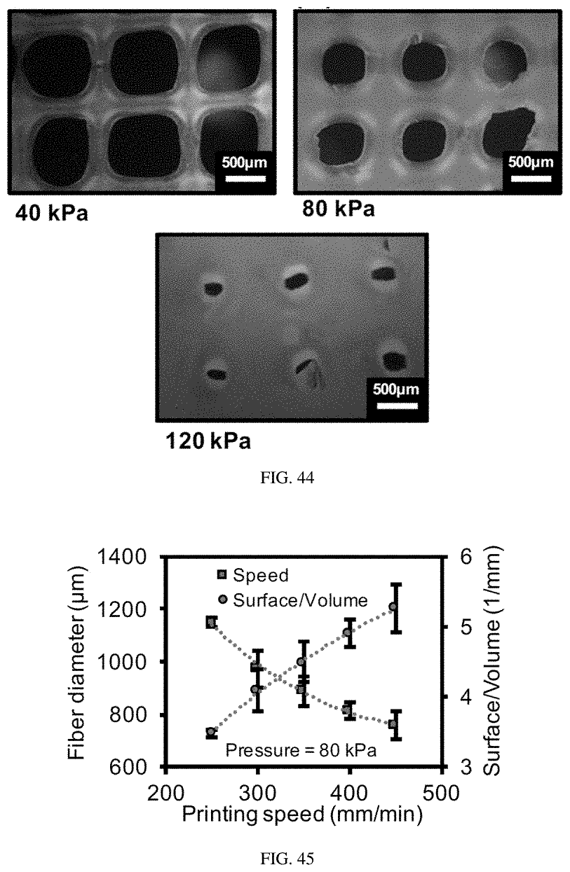

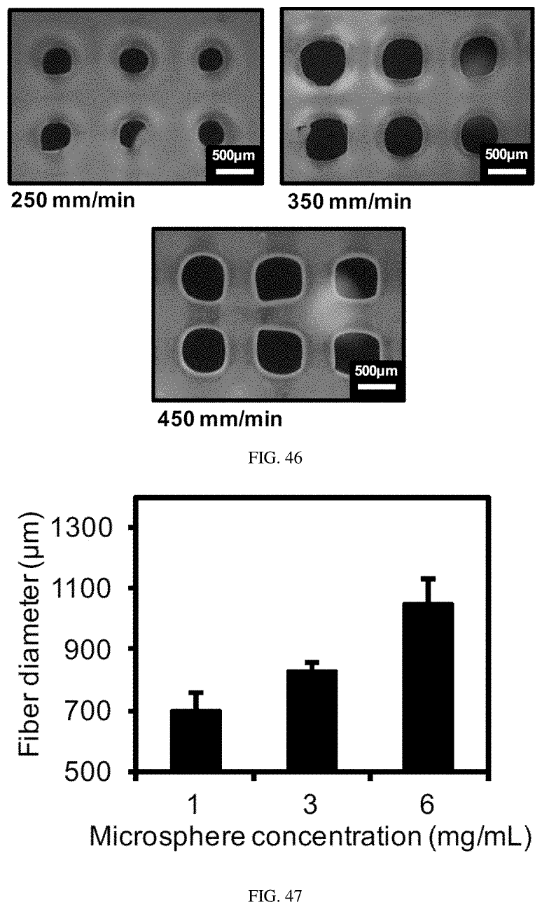

FIGS. 40-42 are photographic and SEM images of one embodiment of the disclosed composition fabricated as a mesh. The photographic images illustrate the suitable porosity and flexibility of the 3D bioprinted construct which allow for nutrients transportation and easy implantation into the brain tissue, respectively. The diameter of alginate fibers were varied to allow manipulation of the surface-to-volume ratio of the construct. This may have a direct effect on the TMZ release kinetics, as a diffusion-based phenomenon. The diameter of 3D bioprinted alginate mesh was characterized by varying the print head pressure, printing speed, and microsphere concentration. The results demonstrated that increasing the print head pressure by 200% contributed to deposition of more alginate from the print head, which in turn resulted in increasing the fiber diameter by 875 μm (FIGS. 43 and 44). However, the fiber diameter reduced by 390 μm when the printing speed was raised by 80%, possibly because less alginate was deposited per unit length of fibers (FIGS. 45 and 46). Fiber diameter (D) is inversely proportional to the surface-to-volume ratio based on the following equation:

$$\frac{Surface area}{Volume} = \frac{4}{D} \qquad Eq . 5$$

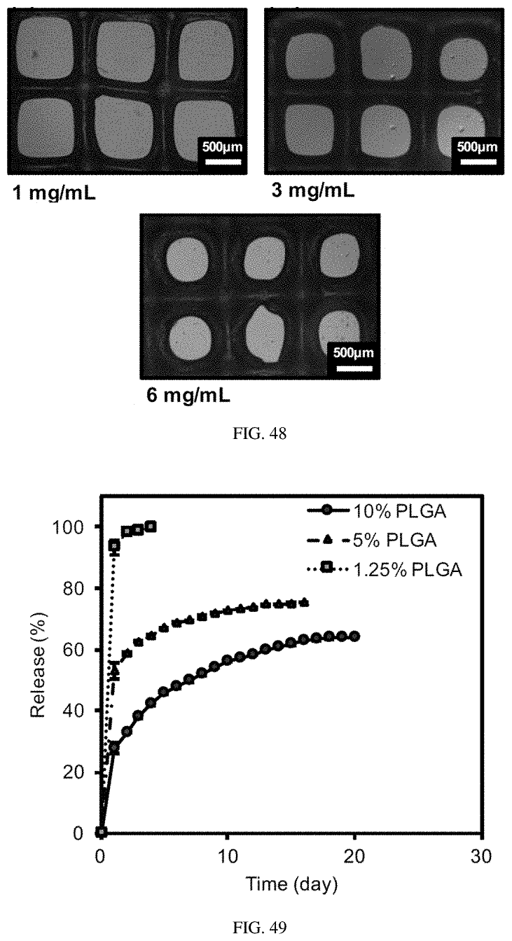

The studies showed that the surface-to-volume ratio of printed meshes decreased by 70% with rising the pressure by 200%, while it increased by 51% when the printing speed grew by 80%. Similar to the print head pressure, FIGS. 47 and 48 demonstrated that increasing the loaded microsphere density from 1 mg mL⁻¹ to 6 mg mL⁻¹ led to 347 μm increase in the fiber diameter.

### In Vitro Release of TMZ

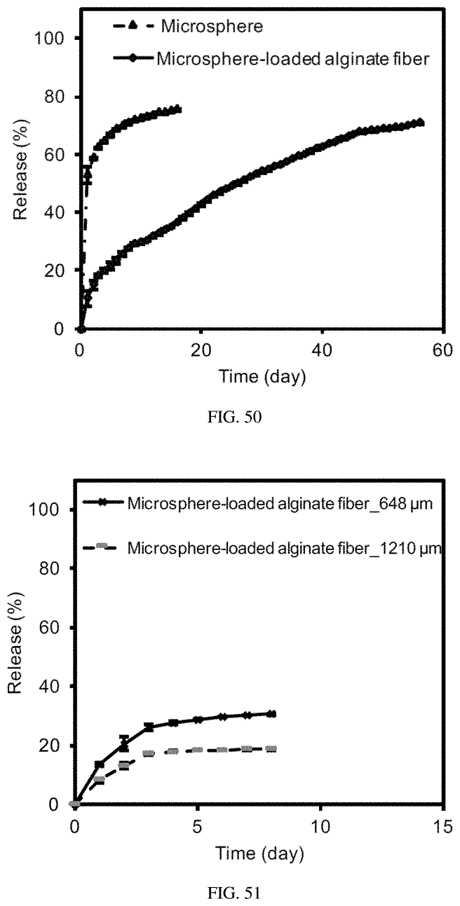

The release of encapsulated drug from PLGA microspheres may occur via any suitable mechanism, such as diffusion, hydrolytic degradation, or a combination of both. It has been shown that the hydrolytic degradation of microspheres may be size dependent and that microspheres larger than 300 μm may undergo a heterogeneous and faster degradation. Since the size range investigated in this study was much smaller than 300 it was hypnotized that the release of the encapsulated TMZ was mostly governed by diffusion. And diffusion can be varied by varying the surface-to-volume ratio of the polymeric microspheres. As described herein, the size of TMZ-loaded PLGA microspheres increased from 7.61±2.74 μm to 27.15±10.04 μm by rising the PLGA concentration from 1.25% to 10% which, in turn, affected the surface-to-volume ratio of microspheres. TMZ-loaded PLGA micro spheres that were prepared with 1.25% PLGA concentration showed an initial burst release of 93% during the first 24 hours and a fast overall release kinetics (FIG. 49). The results demonstrated that TMZ-loaded PLGA microspheres fabricated at higher PLGA concentrations, 5% and 10%, had a lower initial burst release, 53% and 28%, respectively. In addition, the aforementioned microspheres provided a prolonged TMZ release up to approximately 16 and 20 days, respectively. Furthermore, the results demonstrated that the incorporation of polymeric microspheres within wet spun alginate fibers provided a more sustained TMZ release, possibly due to alginate acting as a second barrier against the drug diffusion. As shown in FIG. 50, TMZ-loaded PLGA microspheres prepared with a 5% PLGA concentration and dispersed into alginate fibers, demonstrated a 43% reduction in the initial burst release, compared to similar microspheres not dispersed in alginate fibers, and also provided a prolonged release of TMZ over 56 days.

Additionally, the release kinetics of TMZ from alginate fibers was modified by altering the alginate fiber diameter. Incorporation of TMZ-loaded PLGA microspheres prepared with 1.25% PLGA concentration within alginate fibers with two different diameters showed that by increasing the fiber diameter from 648 μm to 1210 μm, a slower TMZ release rate was obtained, possibly due to the smaller surface-to-volume ratio of thicker fibers (FIG. 51). The diffusion of the drug toward the surface of polymeric microspheres during the evaporation of organic solvent also was considered as one of the major reasons for initial burst release and a fast overall release rate. However, release profiles of TMZ-loaded PLGA microspheres prepared with and without saturation of acetonitrile did not appear to show any substantial difference (data not shown).

### Cytotoxic Effect of the Composition in U87 Glioblastoma Cells

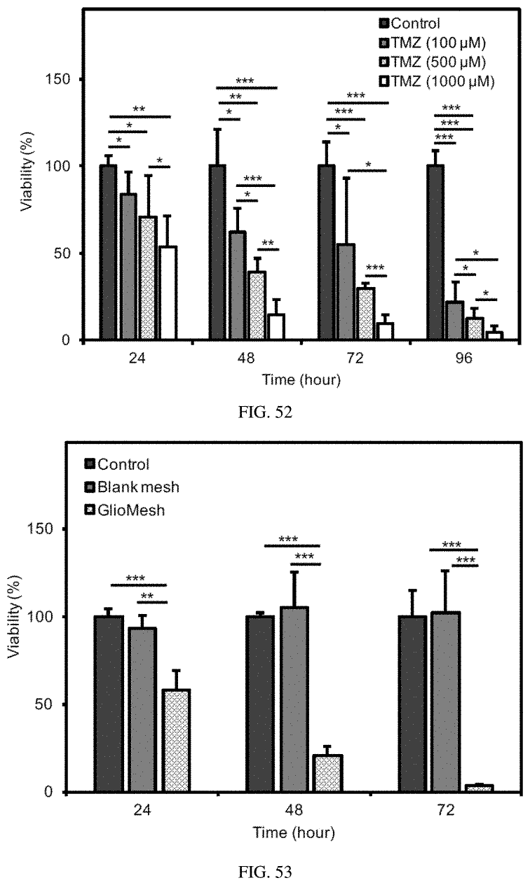

Cell viability tests for U87 glioblastoma cells treated with both free TMZ and microspheres loaded with 100 μM TMZ in alginate fibers were conducted with PrestoBlue™ assay. In the case of 96-hours treatment with the free TMZ, the cell viability reduced by 78% and 95% for 100 μM and 1000 μM TMZ concentrations, respectively (FIG. 52). FIG. 53 shows the cytotoxicity of both blank and microsphere-loaded meshes in U87 glioblastoma cells. Blank meshes did not reveal cytotoxic effects to the cells, whereas the drug/microsphere-loaded mesh substantially inhibited the cell growth. For the microsphere-loaded mesh cell viability experiments, a TMZ concentration of 100 μM was used. As shown in FIG. 53, the viability of U87 glioblastoma cells reached 3% after 72-hours treatment with the drug/micro sphere-loaded mesh. This observation demonstrated the higher cytotoxicity of microsphere-loaded meshes compared to the free TMZ and demonstrated the sustained release of TMZ from the alginate mesh.

### SUMMARY

The (O/O) emulsion method for fabricating the TMZ-loaded microspheres with a high encapsulation efficiency enabled efficient scale-up of this process. Additionally, embedding the microspheres within the 3D bioprinted mesh allows the microspheres to be immobilized at a tumor site, thereby providing an effective localized TMZ delivery. The disclosed drug/microsphere/hydrogel mesh demonstrated sustained release of TMZ over 56 days that can reduce or prevent the need for frequent oral administration of TMZ in GBM patients. The conformational flexibility provided by the alginate allows for a mesh to have conformal contact with irregularly shaped tumor tissue and therefore a microsphere-loaded mesh can achieve a homogenous distribution of TMZ at the tumor site. Furthermore, the mesh facilitates the oxygen and nutrients transportation to the brain tissue after implantation because of its porous structure. And, the cytotoxic activity of the disclosed mesh was confirmed through the extremely low viability observed for the human glioblastoma cells after 72 hours treatment with a microsphere-loaded mesh.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A composition, comprising:
a matrix;
a plurality of microparticles within the matrix; and
a therapeutic agent encapsulated within the microparticles.

2. The composition of claim 1, wherein the matrix is a hydrogel, a synthetic material, or a combination thereof.

3. The composition of claim 2, wherein:
the hydrogel is alginate, gelatin, agarose, hyaluronic acid, gelatin methacryloyl, chitosan, elastin, collage, polyethylene glycol, or a combination thereof;
the synthetic material is poly lactic-co-glycolic acid (PLGA), polylactic acid, polycaprolactone (PCL), nanosilicates, polyvinyl alcohol, poly(n-isopropylacrylamide), or a combination thereof; or
a combination thereof.

4. The composition of claim 1, wherein the microparticles comprise a hydrogel, a synthetic material, or a combination thereof.

5. The composition of claim 1, wherein the microparticles comprise alginate, gelatin, agarose, hyaluronic acid, gelatin methacryloyl, chitosan, elastin, collage, polyethylene glycol, poly lactic-co-glycolic acid (PLGA), polylactic acid, polycaprolactone (PCL), nanosilicates, polyvinyl alcohol, poly(n-isopropylacrylamide), or a combination thereof.

6. The composition of claim 1, wherein:
the matrix comprises alginate, gelatin methacryloyl, or a combination thereof;
the microparticles comprise PLGA, PCL, or a combination thereof; or
a combination thereof.

7. The composition of claim 1, wherein the therapeutic agent comprises an anticancer drug, antiviral, antibiotic, antifungal agent, anti-parasitic, anti-inflammatory agent, pain killer, growth factor, antibody, peptide, cytokine, chemokine, immunomodulatory agent, radioactive composition, clotting aid, or a combination thereof.

8. The composition of claim 7, wherein the therapeutic agent comprises an anticancer agent.

9. The composition of claim 8, wherein the anticancer agent is all-trans retinoic acid, temozolomide, doxorubicin; paclitaxel, 5-fluorouracil, doxurubicin hydrochloride, docetaxel, epirubicin, cisplatin, carboplatin, simvastatin, bevacizumab, ranibizumab, aflibercept, bradikinin, epidermal growth factor, vascular growth factor, interleukin 8, or a combination thereof.

10. The composition of claim 1 wherein the composition is a mesh and the matrix forms fibers that form the mesh.

11. The composition of claim 10, wherein the fibers have a fiber diameter of from greater than 50 to 1000 μm.

12. The composition of claim 11, wherein the matrix further comprises a photo initiator, and the fibers are crosslinked by exposure to UV radiation.

13. The composition of claim 1, wherein the microparticles have a microparticle diameter of from 1 μm to 250 μm.

14. The composition of claim 1, wherein the composition is a mesh composition comprising an alginate matrix with PLGA microparticles dispersed within, the PLGA microparticles encapsulating temozolomide.

15. The composition of claim 1, wherein the composition is a mesh composition comprising an alginate or alginate/GelMA matrix with PCL microparticles dispersed within, the PCL microparticles encapsulating all-trans retinoic acid.

16. The composition of claim 1, comprising:
a first matrix comprising a first plurality of microparticles within the first matrix, and a first therapeutic agent encapsulated within the first microparticles; and
a second matrix comprising a second plurality of microparticles within the second matrix, and a second therapeutic agent encapsulated within the second microparticles;
wherein the first matrix and the second matrix together form a mesh.

17. A method for making the composition according to claim 1, the method comprising:
forming a first mixture comprising the therapeutic agent and a polymer such that a plurality of microparticles encapsulating the therapeutic agent form in the first mixture; and
forming a second mixture comprising the plurality of microparticles and a matrix to form the composition according to claim 1, comprising therapeutic agent-loaded microparticles dispersed within the matrix.

18. The method of claim 17, further comprising forming fibers comprising the composition, and cross-linking the fibers to form a mesh.

19. A method, comprising locating the composition according to claim 1 adjacent to or onto a solid tumor cancer cell in a subject.

20. The composition of claim 19, wherein the cancer cell is a breast, lung, prostate, colon, brain, uterus, pancreas, skin, or liver cancer cell.

21. The composition of claim 20, wherein the cancer cell is glioblastoma multiforme cancer cell.
